(19) United States
(12) Patent Application Publication
(10) Pub. No.: US 2008/0000484 A1
(43) Pub. Date: Jan. 3, 2008

(54) Apparatus for vascular and nerve tissue histogenesis and enhancement

(76) Inventor: Daniel E. Kaiser, Henderson, NV (US)

Correspondence Address: LAW OFFICE OF JAY R. HAMILTON, PLC., 331 W. 3RD ST., NEW VENTURES CENTER SUITE 120, DAVENPORT, IA, 52801, US

(21) Appl. No.: 11/477,175

(22) Filed: Jun. 28, 2006

(51) Int. Cl. A61B 19/00

(52) U.S. Cl. 128/897

(57) ABSTRACT

An apparatus for delivering decompressive energy to soft tissues to stimulate cellular expansion through deep penetration of said applied decompressive energy to said soft tissues to stimulate a predetermined reaction to application of said decompressive energy comprising a vessel having an open end and adapted to encompass the soft tissue to be stimulated. A source of decompressive energy in communication with said vessel and a flexible mass affixed to said open end of said vessel to absorb the pressure exerted by delivery of said decompressive energy to said soft tissue, thereby acting as a seal and force diffuser between said vessel and the soft tissue adjacent the periphery of said vessel.

### CROSS REFERENCE TO RELATED APPLICATIONS (NOT APPLICABLE)

This non-provisional patent application claims priority from and incorporates in its entirety the contents of the provisional patent application previously filed on Jun. 28, 2005 and assigned Ser. No. 60/694,757 by the United States Patent & Trademark Office. This application seeks both United States and International protection for the inventions and inventive methods disclosed herein under both the laws of the United States and the agreed accords of the Paris Convention Treaty (PCT). Patent applications having the following titles and applicant attorney assigned docket numbers are filed concurrently in the United States Patent & Trademark Office and are incorporated by reference herein:

1. USPA0205 “Method for Histogenesis and Enhancement of Tissue”;

2. USPA0210 “Decompressive Thermogenic Bandage”; and,

3. USPA0215 “Selective Destruction of Cancerous Cellular Tissue.”

### FIELD OF THE INVENTION

Vacuum based method (decompressive therapy—DT) and apparatus for treatment of peripheral vascular disease (PVD), Lymphatic, Neuromuscular, bacteriological, host rejection, surgical reattachment of amputated soft tissues, reduction of scar tissue and all other healing/growth response disorders that would benefit from decompressive therapy. Decompressive therapy creates an increase in blood volume and diffusion to targeted tissue (and tissue groups). Decompressive therapy also stimulates the natural creation and transport of growth hormones; responsible for the maintenance and anabolic regenerative tissues of multiple systems including stimulation of the immune system.

As disclosed the present art increases the strength and mass of cell membranes and/or cell walls for therapeutic purposes and repair of function. Additionally, flexibility may be increased for all forms of tissues and/or skin, blood vessels, neurological tissues, glandular tissue, muscle tissues and any form of cellular life that responds to external and internal stress, as is needed.

### STATEMENT REGARDING FEDERALLY SPONSORED RESEARCH OR DEVELOPMENT

No federal funds were used to develop or create the described disclosure.

### REFERENCE TO SEQUENCE LISTING, A TABLE, OR A COMPUTER PROGRAM LISTING COMPACT DISK APPENDIX

(Not Applicable)

### BACKGROUND OF INVENTION

The following description includes information that may be useful in understanding the present invention. It is not an admission that any of the information provided herein is prior art, or relevant to the presently described or claimed inventions, or that any publication or document that is specifically or implicitly referenced is prior art or a reference that may be used in evaluating patentability of the described or claimed inventions.

There has long been an understanding that tissue can and does regenerate in response to application of mechanical force and stress upon the tissue. Orthopedic medicine has long understood the impact that stress has on an area of weakness, i.e. Wolf's Law. For example, any bone(s) under stress, over time, will attract calcium salts which will fuse it to the surrounding bones as a protective measure to resolve the stress or weakness. The body also reacts to the application of abnormal stress. During pregnancy, for example, nature provides for the expansion of the skin (and other parts of body) to accommodate internal growth including subcutaneous growth both the fetus and mother, as well as weight loss and/or gain.

Prior art devices and methods include surgical techniques wherein balloons and external and/or internal fixation pins are inserted into the body for limb lengthening. See U.S. Pat. No. 5,074,866 issued to Sherman et al. for “Translation/Rotation Device for External Bone Fixation System,” incorporated by reference herein, for further discussion of this area of the prior art. The general background for this area is further set forth in U.S. Pat. No. 5,536,233 issued to Khouri for “Method and Apparatus for Soft Tissue Enlargement” as the basis for the improvement described therein. (Hereinafter referred to as “Khouri”.) The generalized method and apparatus described in Khouri is an improvement over the prior art and describes the general basis for the improved invention described herein. As noted in Khouri, the prior art failed to achieve long term soft tissue enlargement without damage to the soft tissue being enlarged, as well as the surrounding tissue. This damage to the surrounding tissue has limited the amount of vacuum which may be applied to the soft tissue for purposes of enhancement or enlargement. Khouri has attempted to avoid this damage to surrounding tissue by the use of a rim around the periphery of the dome to which the vacuum is applied. This rim is described as having sufficient surface area so that the pressure applied by the rim is less than or equal to the negative pressure applied to the soft tissue under the dome. By regulating the pressure within the dome to 1.5 inches of Mercury (Hg), the damage to the soft tissue is avoided by use of the rim. The prior art is limited to a vacuum with a magnitude of less than 1-1.5 inches of Hg which limits the enhancement. The prior art also uses a band of adhesive applied to the seal to allow it to physically stick to the skin of the individual wearing this invention. The daily use of this device has been shown to cause contact dermabrasion which can leave scars as well as break the skin, increasing susceptibility to infection. Other examples of prior art along this line include U.S. Pat. Nos. 6,500,112; 6,478,656; 6,355,037; 6,309,394; 5,704,938; 5,701,917 and 5,695,445; 5,676,634; and 5,662,583, which are all incorporated by reference herein.

Other important art in this area includes U.S. Pat. No. 6,042,537 ('537) issued to Kaiser for “Apparatus and Method for Tissue Enlargement” incorporated by reference herein and hereinafter simply referred to as “Kaiser,” which teaches a vacuum apparatus having a dynamic load bearing diffusion seal. The seal as taught by Kaiser in '537 allows for and dynamically absorbs, transfers and directs the dynamic static loads placed upon it to a safe and effective equilibrium. Kaiser teaches a force diffusion seal primarily for loads wherein the plane of the tissue treated is substantially perpendicular to the apparatus vessel walls. Kaiser is an improvement over the cited prior art and is adequate to handle dynamic loading of static forces of this nature. New types of dynamic loads are created by the apparatus, method and process disclosed and claimed herein. The present application requires a diffusion seal capable of handling a plurality of dynamic loads that may be delivered from opposite directions.

The normal animal cell, including that of humans, has in general a predefined shape and size. It has been discovered when sufficiently stressed, the cell will increase in size and its external structure will also deviate to accommodate most any vacuum or negative force that is applied to the cell. Proper application of decompressive energy (such as by vacuum force) to the cellular structure can induce the cell to replicate and/or accommodate the stress that is applied by the decompressive energy. The resiliency of cellular membranes and supporting structures, as noted in the prior art, can be damaged beyond repair by the improper application of an excessive amount of decompressive energy. The amount of decompressive energy applied should be properly controlled and limited both manually and automatically to avoid damage to both adjacent and treated tissues, including their internal mechanisms and membranes.

As noted above, the prior art devices have failed to achieve long term soft tissue enlargement while preventing damage to the soft tissue being enlarged, as well as any surrounding tissue. These prior art devices have not been successful because the amount of vacuum necessary to provide successful enlargement of the soft tissue has not been able to be achieved without damage to surrounding tissue. The low vacuum pressure described in the prior art does not provide for adequate enhancement or enlargement of the soft tissue because the amount of pressure was limited by the ability of the device to prevent damage to the surrounding tissue.

This invention has shown that animal cellular structures can accommodate vacuums from 0.0009 inches of Hg to 30 inches of Hg. It has been found that the optimum decompression energy through vacuum force (in inches of Hg) necessary to produce the desired affect of inducing cellular reproduction due to stimulation of and the release of HGH (human growth hormone) and/or cellular strengthening through hyper-enhancement of the soft tissues immune system responses is approx 8-10 inches of Hg. Clearly, tissue enhancement can be achieved at lower or higher decompressive energy levels. It is contemplated that a range of values may be applied that are both less than 8 inches of Hg and greater than 10 inches of Hg to provide a desired response. Improperly applied lower pressures and stresses if not used in accordance with this invention and its method of operation may also cause cellular damage. It is theorized, however, that if the body's tissues are stimulated properly and the methods are applied in accordance within tissue limits and with this invention that even higher forces and stresses might safely be obtained.

The body's immune system can routinely repair most, if not all, damage caused by minimal to medium amounts of vacuum applied to healthy tissues. This is similar to the repair of minor contusions, discoloration and vascular seepage caused by small amounts of vacuum such as that which can be applied to the skin by the vacuum induced by the mouth.

As disclosed by the prior art, tissue enhancement and histogenesis by means of vacuum does in fact occur. However, the prior art is limited in application to the breasts and the penis. Additionally, the prior art does not teach a method or apparatus capable of applying increased amounts of vacuum or negative pressure to living tissues without damaging surface or upper layers of tissue to increase circulatory response or cellular enhancement.

Given the weakness and limitations of the prior art, what is needed and desired is a safe, non-invasive method of tissue histogenesis for skin, vascular tissues, neurological tissues, glandular tissues, muscle tissues and any other form of cellular life that responds to applied external and internal stresses for the treatment of many disorders including many peripheral vascular diseases. A safe way to increase the strength and mass of cell membranes and/or cell walls for therapeutic as well as repair of function and flexibility to all forms of tissues and/or skin, blood vessels, neurological tissues, glandular tissue, muscle tissues and any form of cellular life that responds to external and internal stress is also needed. Additionally, a safe apparatus and method are needed to stimulate the natural immune system response along with tissue repair and formation as discussed above. The prior art fails to provide a diffusion seal capable of handling the dynamic loads created by the specific applications and processes for vascular and nerve tissue histogenesis and enhancement disclosed and claimed herein.

### Peripheral Vascular Disease Physiology (Background)

All tissues of the body require oxygen and nutrients to survive. Transportation for these two necessities rests solely on the vascular network. Arterial disease can affect the body systemically; however, the peripheral network in the extremities is normally first to be symptomatic. Restoration of blood flow is critical or tissue function deteriorates. Failure to restore vascular integrity results in pain (lactic acidosis) and finally tissue apoptosis—quickly moving on to skin ulcerations, infections and eventually gangrene which will require amputation of the diseased extremity. Amputation however, does not address the need to restore blood flow to the remaining tissue. An understanding of peripheral arterial disease requires knowledge of vascular structural elements and their arrangement within vessel walls. Vessels beyond a certain lumen diameter generally consist of three defined layers: the intima, media, and adventitia. See Talbert R L. Peripheral vascular disease. In: DiPiro J T, Talbert R L, Hayes P E, Yee G C, Matzke G R, Posey L M. Pharmacotherapy: A Pathophysiologic Approach. Norwalk, Conn.: Appleton & Lange, 1993: 388-400. The intima is a single layer of endothelial cells on the innermost section of the vessel wall. Media refers to the middle section of the vessel wall and consists of smooth muscle cells surrounded by collagen and elastic tissue. Adventitia, the outermost covering of the vessel wall, consists of a mixture of collagen, elastic tissue, smooth muscle, nerve fibers, vaso vasorum, and lymphatic vessels which accommodate lymphatic flow to nourish and remove metabolic waste products from the vessel wall. See Spittell P C, Spittell J A. Managing combined peripheral and coronary artery disease. Contemp Intern Med 1993 (September). The structural elements most common to arterial vessels consist of five separate tissue components: endothelium, basement membrane, elastic tissue, collagen, and smooth muscle. The endothelium comprises a flat layer of endothelial cells lining the entire vascular system. Below the endothelium is the basement membrane, composed of various proteins and polysaccharides which serve as a support structure and transport medium for various materials. Elastic tissue encompasses the endothelium and basement membrane. Collagen, a major protein of the white fibers of connective tissue, cartilage, and bone, resists stretching and thereby prevents over distension of the vasculature. Smooth muscle provides the contracting component of the vascular system that regulates vasoconstriction and dilation. It has been known for some time that the peripheral pressure pulse contains information on arterial stiffness and vascular tone and that increased arterial stiffness correlates with increased risk of a major cardiovascular event. The specific validation of Pulse Trace was done at St Thomas' Hospital and has been published. These papers demonstrated a simple linear relationship between the shape of the Digital Volume Pulse and that of the peripheral pressure pulse which remains constant irrespective of the effects of hypertension or effects of vasodilatation produced by NTG, and that the Stiffness Index (SI) parameter correlates with PWV, the gold standard for arterial stiffness. When the vascular system has been compromised (not including trauma induced) there is a cascade effect that, if left unchecked, will continue to deteriorate and starve healthy tissue. Several historical methods of blood restoration to tissue have been attempted and of these methods, surgical and pharmacological remain the most widely accepted. Surgical methods and procedures are similar to a coronary bypass, the procedure to correct or “bypass” a damaged vessel involves the surgical attachment of a synthetic tube or sewing on a segment of healthy vein donated from another area of the body. Blockages in diabetics may occur further down the leg and may require a bypass to an artery such as the posterior tibial or dorsalis pedis. Surgery is generally effective for limited correaction each time the surgery is performed. However, it requires a patient in fair health to handle the general anesthesia required for this type of procedure and the same systemic problems that impacted the vessel to begin with will, over time, begin to work against the surgically corrected segments.

### Peripheral Vascular Disease Physiology in Relation to Diabetic Neuropathy

Vascular compromise is one of the key factors for Diabetic Neuropathy. Nerve tissue is reliant on adequate blood flow to provide nutrients to the tissues and remove metabolic waste. Normally, capillaries facilitate the passage of nutrients into the cell and permit the removal of waste products into the bloodstream. Hyperglycemia will create a less permeable wall which, over time, allows for a buildup of toxic metabolites. The buildup will eventually impact cellular metabolism. When adequate blood flow to nerve tissue is not available to perform these functions, vascular damage and dysfunction of the nervous system can occur.

The risk of lower limb amputation in patients afflicted with diabetes is 15 to 40 times higher than in those without diabetes. Ulceration of the foot is often the initiating lesion leading to amputation. See Pecoraro R E, Reiber G E, Burgess E M. Pathways to diabetic limb amputation: basis for prevention. Diabetes Care. 1990; 13:513-521. Diabetes 1996 Vital Statistics. Alexandria, Va.: American Diabetes Association, 1996:1-102. AD—Section of General Medicine, Veterans Affairs Medical Center, Oregon Health Sciences University, Portland. Diabetic patients are particularly vulnerable to foot ulceration due to the coexistence of peripheral neuropathy and peripheral vascular disease. Peripheral sensory neuropathy is the single most common contributory factor leading to the development of ulcers in the feet of people with diabetes, accounting for up to 87% of new ulcers. See Boulton A J M. The Diabetic foot: Neuropathic in Aetiology? Diabet Med. 1990; 7:852-858. The first examined cause is a length-dependent “dying back” axonopathy, primarily involving the distal portions of the longest myelinated and unmyelinated sensory axons, with relative sparing of motor axons. The morphologic characteristics of diabetic polyneuropathy are consistent with either a vascular or a metabolic cause of the problem.

Patients with intermittent claudication experience pain or cramping even when they are only resting; and especially for those patients with ulcers that are persistent in not healing, little hope remains for improvement unless a new source of blood can be provided to the affected limb.

The apparatus and methods claimed and disclosed herein are considered a potential means for the reversal of neuropathy and the effects of peripheral vascular disease (PVD). It is believed that the increase in peripheral and deep blood flow to the tissues in the extremities should have a positive impact on the basement membrane of the blood vessels and increase not only blood flow but also transfer of nutrients and waste products from the tissues previously affected. Upon restoration of the vascular network, there is induced a wound healing response wherein blood vessel histogenesis (vascular tissue generation or regeneration) can occur.

It is also well known that diabetes has a major impact on the nervous system. Statistics and studies suggest that 60 to 70% of persons having diabetes suffer mild to severe symptoms from attendant nervous system damage. Symptoms may include impaired sensation in the feet and/or hands, pain in the feet and/or hands, slowed digestion of food in the stomach, Carpal Tunnel Syndrome and other nerve problems. Studies and statistics suggest diabetic neuropathy is a causative factor in more than 60% of the non-traumatic lower-limb amputations in the United States. It is an objective of the present apparatus and methods to provide restorative effects upon the vascular network and further induce a wound healing response in the impaired nervous system wherein nervous tissue histogenesis (generation or regeneration) can occur.

Diabetic neuropathies can be classified as peripheral, autonomic, proximal, and focal. Each affects different parts of the body in different ways. Peripheral neuropathy causes either pain or loss of feeling in the toes, feet, legs, hands, and arms. Autonomic neuropathy causes changes in digestion, bowel and bladder function, sexual response, and perspiration. It can also affect the nerves that serve the heart and control blood pressure. Autonomic neuropathy can also cause hypoglycemia (low blood sugar) unawareness, a condition in which people no longer experience the warning signs of hypoglycemia. Proximal neuropathy causes pain in the thighs, hips, or buttocks and leads to weakness in the legs. Focal neuropathy results in the sudden weakness of one nerve, or a group of nerves, causing muscle weakness or pain. Any nerve in the body may be affected. Neuropathy is a very disturbing consequence of low blood flow states. Different widely know generalized diseases result in neuropathy, such as diabetes. By restoring blood flow, neuropathy may decelerate progression of disease.

### SUMMARY OF THE INVENTION

A medical apparatus and methods for the treatment of peripheral vascular disease (PVD) and other medical disorders and ailments that would benefit from increased and enhanced tissue response due to increases in blood flow on both a macro and micro vascular level including increased cellular stimulation and response is disclosed and claimed herein.

Furthermore, the maintenance of a constant or static negative pressure (vacuum) combined with a continuous dynamic vacuum circulation or recirculation of energy within the unit produces a dynamic micro energy gradient. This dynamic micro energy gradient creates an inducing or directing flow. This energy gradient results in a mass transfer gradient. Thus, allowing the circulation of blood flow to be controlled or directed to areas of the tissue with the greatest resistance to blood flow. Selection of decompressive energy source (such as vacuum), vessel shape and alternating decompressive/non-decompressive force regimens may further optimize the dynamic vacuum circulation of energy.

As disclosed, the present art is a novel technology and method for application within the medical technology as well as the biological technology fields. The disclosed concepts revolve around the application of decompressive energy or vacuum forces to different elements as well as form, function and homeostasis affecting the cellular biology, neurology, immunology and vascular tissues of humans and animals. Some symptomatic ailments this technology may treat or alleviate are symptoms associated with diabetes and arthritis. Included herein are the device designs and methodology for the treatment of PVD's (peripheral vascular disease) reduction in blood flow and nerve degeneration symptomatic of human diabetes for the hands and feet.

The technology has many other therapeutic uses including immune system enhancement, cellular development, vascular and neurological system development or regeneration and even possible organ regeneration on some levels. This technology has proven to be effective in controlling the growth of infectious agents and organisms.

As disclosed, the components of the technology include the design of the vessel, the application of the dynamic seal between the vessel and the tissue and/or sub terrain tissue to be treated and the method of treatment of the tissue.

This invention produces a permanent enhancement of tissue, especially soft tissue, without surgical or other deleterious effects on the patient. This invention overcomes the restriction of limiting the negative pressure which may be utilized for cell enhancement by diffusing the contact loads and stresses by using a novel seal, which also overcomes the excessive pressures that previously would have been applied to the surrounding tissue causing crushing and/or cellular tissue damage. This invention allows for the controlled development of increased blood flow deep inside the human body. The method and apparatus disclosed and claimed herein allow the delivery of mechanical force in a safe and non-invasive way deep within the body to stimulate the natural healing mechanisms and the body's ability to maintain a homeostasis state.

When this method and apparatus is initially used at vacuum of 1-9 inches of Hg, at the beginning of the hyper-enhancement process, small and superficial contusions or bruising may occur. It has been determined that the comfort level of vacuum should be gradually increased over a period of time, starting from approximately 1.0-1.5 inches of Hg and proceeding to higher values of vacuum and decompression. The apparatus upon which tests were conducted would create a vacuum that was the maximum allowable on and inside earth's biosphere. This maximum amount was reduced for greater safety to the subject.

A Phase 1 Study has been designed and approved for use with the apparatus and methods disclosed herein. See “A Study to Document the Effect of a Novel Device Employing Negative Pressure to Increase Vascular Flow and Diffusion in the Extremities.” The objective of the study is to demonstrate the ability of decompressive energy to raise the vascular flow and diffusion of blood supply to the extremities. During the study, the effects on the elasticity of blood vessels (endothelial testing) as well as nerve conduction testing will be monitored. As designed and approved, the eight subjects will be treated for up to five (5) minutes with a range of negative pressures on one or both arms (alternating—not simultaneously). Normal values will be established for pre and post treatment as well as neurological impact, and pre/post skin condition. This study upon completion will provide the basis for a Phase 2 Study which will apply the methods and apparatus disclosed herein to subjects with diagnosed levels of peripheral vascular disease symptomatically present in the extremities, with a special emphasis on diabetes.

One facet of the vessel design is that it has a specialized flexible vacuum seal having properties that allow the seal, by design, to handle or absorb and/or instantaneously transfer, whether directly and/or indirectly, such dynamic forces and dynamic loads and/or dynamic stresses, as applied to the tissue or in other words “diffuse” the dynamic stresses and forces virtually instantaneously. This specialized vacuum seal, by its very design, dynamically reduces the normal crushing restriction of blood flow, and/or dynamically reduces the normal contact pressures and/or stresses and forces that are delivered to the contact points of the vacuum seal's contact material, and the living tissue contact areas at the point and/or place of contact with living tissue and the tissues surrounding and under laying the tissues directly affected by treatment.

Another facet of the vessel design is that it can be constructed of any transparent and/or opaque material that is so engineered and/or designed to withstand vacuum or negative pressure and/or decompressive energy within said vessel to a value of up to 30 inches of mercury (Hg).

The device as designed can be made of many interlocking sealing segments and/or come as a custom molded unit that is patient specific. Some applications will require customization of the vessel and others will not. The design of the vessel will be determined by the needs of the patient and/or the specific treatment area and/or the therapy necessary to stimulate the desired tissue response (i.e. tissue growth), vascular regeneration, neural network regeneration, increased blood flow, pharmaceutical delivery and selective destruction of diseased or malignant cells.

The system as envisioned and designed includes a dynamic pump that has sufficient volume to create a desired level of vacuum up to inches 30 Hg in a desired specific amount of time which may range from as little as a nanosecond to hours.

The system as envisioned and designed includes a control system that can be pre-programmed and/or permanently and/or semi-permanently programmed to allow for specific vacuum loads and application times, or any combination therein to be achieved by the system. The control system as envisioned and designed allows for a combination control of the following system variables:
1. Time to peak vacuum (mm Hg) flow;
2. Peak vacuum (mmHg) to be maintained for a predefined amount of time;
3. Controlled release of dynamic vacuum inside the device vessel;
4. Controlled rest periods i.e. periods without application of vacuum and/or reduced vacuum;
5. Automatic programmable functions;
6. All necessary control sensors to analyze environmental factors;
7. All necessary control sensors to analyze stimulation variables;
8. Sensors to provide data on interior and/or exterior environments of said vessel while vacuum chamber of said vessel is both under actual vacuum conditions and not. The sensors can also provide data inputs for, but are not limited to, temperature, humidity, sound/sonic, blood pressure, ambient atmospheric pressure, tissue density, measure by ultrasound, sonar, or any form of sounding device, or any frequency of light and/or radio signal or carrier wave, electrical resistance test to measure cellular conductivity of electrical impulses and/or current flow.
9. The control system also allows control functions to be utilized individually and/or in combination with other control functions.
10. The control system also allows control during application for the depth of tissue penetration of the vacuum energy.
11. Finally, the device as described and shown should be comfortable for patient to wear and use, as well as being easy to use, operate, maintain and sanitize.

Finally, another attribute of the technology as described and disclosed herein is the application of the vacuum or vacuum energy to the tissue itself. The normal animal cell, including that of humans, has in general a predefined shape and size. It has been discovered when sufficiently stressed, the cell will increase in size and its external structure will also deviate to accommodate any vacuum or negative force that is applied to the cell. Proper application of vacuum to the cellular structure can induce the cell to replicate and/or accommodate the stress that is applied by the vacuum. The resiliency of cellular membranes and its supporting structure, as noted in the prior art and as discovered in the use of this invention, can be damaged beyond repair by the improper application of an excessive amount of vacuum. Therefore, the amount of vacuum applied must be properly controlled and limited, either manually or automatically, to avoid damage to the tissues, including their internal mechanisms and membranes.

It has been shown that animal cellular structures can accommodate vacuums from 0.0009 inches of Hg to less than or equal to 30 inches of Hg without massive destruction of tissue, if properly applied. Vacuum at most any level of Hg may cause damage to cells if the proper application and methodology is not applied and cells are not allowed to properly acclimate to the applied stresses caused by the vacuum. It is also known that improperly applied vacuum even at lower negative pressures may also cause tissue and cellular damage as with the prior art. Improperly applied rapid decompression (applied vacuum) can destroy most soft tissue cells. The body's healthy immune system can routinely repair most, if not all, light damage caused by vacuum's decompressive energy.

This invention has indicated that the optimum pressure or the optimum vacuum in inches of Hg necessary to produce the desired affect of inducing cellular reproduction or cellular strengthening through hyper-enhancement of the soft tissues immune system responses will depend on what one wants to do and what type of cellular matter is being worked with. Neurological versus connective tissue will respond in drastically different ways to decompressive/mechanical forces generated by vacuum energy. It is possible, however, that there are generalities that can be applied to tissue groups, organs and individual tissues needed to provide a desired response.

As a result of experiments utilizing this invention, it has been recorded that each new generation of cellular growth or enhancement improves the elasticity, toughness and health of the cell membranes. Observations of the experiments of applicant indicate that the longer cell structure is stressed by applying 25-75% of the safe maximum vacuum in inches of Hg over an extended period of time, new cellular growth is stronger in structure and more resilient. It has also been shown from the experiments that the greater the decompression of cellular matter, the greater the benefits; if properly applied through time with proper pressure and vacuum chamber design.

Continuously or semi-continuously applying the vacuum energy to and into the tissues, as controlled by the system programming, stimulates a dynamic response from the biological mechanisms of the living tissue, one such predicable response is the dramatic increase in blood flow. Another such response is the development of micro-vascularization. By operating the system in this manner, function of the vacuum device may be alternated to stimulate many other predictable events of the bio mechanisms of the living tissues. Another function of the system controls and methods of operation is that the system may be optimized for either tissue generation, regeneration or enhancement. The basic formula criteria or variables needed for treatment or stimulation of tissue for enhancement are:
1. Type of tissue;
2. Health of tissue;
3. Gradient or depth of tissue;
4. Amount of decompressive energy to be delivered to the tissue to be treated;
5. Surface loads of the decompressive energy needed to penetrate to the desired depth;
6. Requirement for positive pressure augmentation via compression wrap;
7. Speed of cellular hydration (edema);
8. Recovery time for reclamation of excess fluids in treated cellular tissue;
9. Amount of decompressive energy to be applied;
10. Time of decompressive energy application;
11. Need for incremental increase of application time, decompressive energy applied and positive pressure applied via a compression wrap;
12. Patient compliance;
13. Ability to monitor improvements; and,
14. Patient comfort.

This invention overcomes the prior art's limitation of limited amounts of negative pressure (vacuum) which may be utilized without tissue damage. This invention, though noninvasive, allows for the controlled increase in sub-dermal blood flow as well as the potential for controlled diffusion of energy to depths in excess of three (3) centimeters inside the human body. The method and apparatus disclosed and claimed herein allow the non-invasive, safe delivery of decompressive energy, through mechanical forces or other means, deep within the body to stimulate the natural systems that are responsible for corporeal repair, regeneration and homeostasis.

The higher levels of decompressive energy (through vacuum forces) can only be applied safely by diffusing the contact loads and stresses generated through application of vacuum to the tissues as disclosed and claimed herein. One benefit of this invention is the controlled development of increased blood flow deep inside the human body, as well as an increase in micro-vascularization throughout the treatment area. Vascularization is the organic process whereby body tissue becomes vascular and develops capillaries

When this method and apparatus is used within a range of 0.0001-9 inches of Hg, at the beginning of the hyper-enhancement process, small and superficial contusions or bruising may occur. It has been determined that the comfort level of vacuum should be gradually increased over a period of time, starting from approximately 1.0-1.5 inches of Hg (depending on tissue to be treated) and proceeding to higher values of vacuum and decompression. The apparatus previously used for testing created a vacuum that delivered the maximum allowable decompressive energy (vacuum) within the earth's atmosphere. This maximum amount was significantly reduced to safe levels when applied to the subject.

This invention has also been utilized with variations in the configuration of the dome, sphere, or shape of a vacuum applicator and/or containment vessel. Varying the shape of the vacuum applicator varies the forces exerted upon and into the material or tissue exposed to vacuum energy. Thus, the tissue may be elongated, lengthened, or widened by enhancement or expansion within and in conjunction with the sphere.

It has also been discovered in the use of the invention that the more tissue under and in proximity to the dome increases the dynamic forces and the rate of tissue enhancement and hyper-enhancement. Thus, this invention provides for a plurality of vessels or domes with various configurations to control the direction and the rate of cellular enhancement or enlargement. Additionally, this invention provides for a plurality of vessels or domes with various configurations to control the depth that decompressive energy can penetrate into the body of the subject and the amount of decompressive energy delivered to the surface of the skin and/or deep inside the tissue or tissues being treated.

The decompressive energy (through vacuum force) acts to cause the veins and arteries to enlarge and engorge, facilitating the benefits of increased blood flow, which is a beneficial side effect provided by this invention in conjunction with tissue growth. Although this invention has not been utilized, except to produce new and enhanced or enlarged soft tissue structures, it is believed that other uses of vacuum pressure to induce cellular growth and immune system hyper-enhancement would be useful in other areas and medical applications and treatments that would benefit from this type of predictable dynamic energy.

The increase in blood flow, due to enlargement and/or enhancement of healthy and normal blood vessels, is of substantial benefit through the increase in malleability, strength and overall health of the vessels themselves. The increase in blood flow would, over time, improve the surrounding cells and provide more nutrients to damaged areas to aid in the repair of wounds and/or unhealthy tissue that lacked proper oxygen levels. Research and experimentation both by the medical community and inventor suggest the method and apparatus disclosed herein may be useful on most any tissue that has morphemic characteristics.

This invention allows the use of a method used to enclose soft tissue within a transportable containment device, applying specific and substantial controlled vacuum to decompress soft tissue. The development of new vessels or instruments, which could enclose the area or tissues to be repaired and provide appropriate decompressive energy (vacuum force) while not damaging the surrounding tissue, are disclosed and claimed herein.

As noted above, the prior art devices have failed to achieve long term soft tissue enhancement while preventing damage to the tissue acted on, as well as any surrounding tissue. These prior art devices have not been successful because the amount of vacuum necessary to successfully create or stimulate the tissues has been limited by the potential for damage to surrounding and supporting tissues.

This invention allows application of larger amounts of decompressive energy (through vacuum force or negative pressure) to be applied to specific tissues, under substantial control, to decompress tissue within a containing device or vessel without damaging surrounding supporting tissues for the enhancement of the tissue within the vessel.

The downward force created by the vacuum inside the vacuum chamber is absorbed by diffusion of forces applied and generated through the vacuum seal without damage to the surrounding tissue against which the container reacts. Therefore, this invention is able to use a vacuum pressure which delivers sufficient decompressive energy to create distraction force in adequate supply to facilitate the enlargement, enhancement, stimulation of growth hormone production, increase of blood flow, strengthening of cellular membranes, stimulation of new cellular development, increase of immune system response, stimulation of neurological regeneration and many other positive and predictable responses to targeted soft tissues at greater decompressive energies (vacuum pressures) than prior art devices.

The novel seal and force diffuser between the vacuum chamber and the human cells or tissues surrounding the tissues to be enhanced permits the use of a dynamic vacuum force which will stimulate cell activity without permanent harm to cells and/or user. The force diffusion seal of the apparatus disclosed and claimed herein allows dynamic handling and control of loads delivered to the bottom surface of the force diffuser seal and loads emanating from inside the force diffuser seal (upward and inside out). These new types of dynamic loads are created specifically due to the nature of the application and process for cellular and/or tissue enhancement as generally disclosed and claimed, and specifically for the methods and apparatus for treatment of peripheral vascular disease and tissue histogenesis and enhancement.

It has also been demonstrated that the total destruction of the healthy cell membrane and the nucleus by stretching or elongating beyond there physical limits through application of mechanical forces will destroy these cells. Unhealthy cells, however, are proven to be less resilient and can be destroyed at different pressures or forces, thus providing a selective advantage with application of greater decompressive pressures. This provides dual health benefits through the potential destruction of unhealthy cells and enhancement of healthy cells. Some unhealthy cells will be destroyed with even small amounts of vacuum (decompression). This effect may have beneficial effects in the controlled targeting of diseased tissues that need to be eliminated for medical reasons to benefit the patient. This difference in mechanical properties between healthy and unhealthy cells provides an opportunity alone or in combination with the delivery of beneficial compositions to exploit these differences to the benefit of the tissue treated.

### OBJECTS OF THE INVENTION

It is therefore an object of this invention to disclose and claim the apparatus and methods for the treatment of peripheral vascular disease (PVD) and other medical disorders and ailments that would benefit from increased and enhanced tissue response due to increases in blood flow on macro-vascular, micro-vascular and a collateral level including increased cellular stimulation and response.

It is therefore an object of the present invention to provide a method and apparatus to stimulate and improve tissues which may be non-invasive.

It is still another object of this invention to stimulate increased blood flow in vascular systems.

It is a further object of the invention to provide a system and method that allows for deep penetration of decompressive energy into human or animal tissues.

It is still another object of this invention to provide a method and technology that stimulates tissue and cellular growth.

It is therefore an objective of the invention as disclosed and claimed to allow treated tissue to be affected by decompressive energy to be placed inside and/or underneath the vacuum device.

It is another objective of the invention as disclosed and claimed to allow placement of apparatus on or around a body part to affect treatment.

It is another objective of the invention as disclosed and claimed to allow insertion of the decompressive energy or vessel into the body or body part for engagement with the tissue to be treated by decompressive energy.

It is another objective of the invention to use vacuum as a decompressive energy.

It is another objective of the invention as disclosed and claimed to control the application of decompressive energy by contouring or shaping the vessel in such a way as to modulate the response of the tissue to the vacuum to affect the desired change in therapeutic application and to control stimulation rate, growth rate and/or blood flows.

It is still another object of this invention to provide a method and technology that stimulates the strength, flexibility, and expandability of tissues and/or cellular membranes and internals.

It is still another object of this invention to provide a method and technology that stimulates increased blood flow, vascular elasticity and permeability. U.S. Pat. No. 6,503,205 issued to Manor et al. for “Dual Ultrasonic Transducer Probe for Blood Flow Measurement, and Blood Vessel Diameter Determination Method” is incorporated by reference herein for further background in analytical methods and apparatus available to those skilled in the arts.

It is still another object of this invention to provide a method and technology in controlling loads delivered onto both the bottom and side surfaces of the force diffuser seal through loads emanating from inside the force diffuser seal (upward and inside out).

It is another object of the invention to provide a control system for the method and apparatus disclosed herein for improvement of cellular tissues that may be controlled manually or be automated for computer control and data collection.

It is another object of the invention to use the methods and apparatus disclosed herein with pharmacological compositions beneficial to vascular elasticity, vascular permeability, vascular angiogenesis and vasculogenesis. U.S. Pat. No. 6,713,065 issued to Baron et al. for “Methods of Using Hedgehog Proteins to Modulate Hematopoiesis and Vascular Growth” is incorporated by reference herein for pertinent background on the nature of vascular angiogenesis and vasculogenesis. U.S. patent application filed by Coleman having publication #20060057117 and entitled “Vascular Endothelial Growth Factor 2” relates to compositions useful in stimulating wound healing and vascular tissue repair and is incorporated by reference herein.

It is another object of the invention to use the methods and apparatus disclosed herein with nano devices, such as nano-cells and nano-shells, for improved delivery of pharmacological compositions beneficial to vascular elasticity, vascular permeability, vascular angiogenesis and vasculogenesis. U.S. Pat. Nos. 6,645,517, 6,530,944, and 6,428,811, issued to West et al. for “Temperature-Sensitive Polymer/Nanoshell Composites for Photothermally Modulated Drug Delivery”; “Optically-Active Nanoparticles for Use in Therapeutic and Diagnostic Methods”; and “Temperature-Sensitive Polymer/Nanoshell Composites for Photothermally Modulated Drug Delivery,” respectively, are incorporated by reference herein.

It is another object of the invention to use the methods and apparatus disclosed herein with nano devices, such as nano-cells and nano-shells, for improved delivery and/or actuation of pharmacological compositions beneficial to the destruction of diseased, malignant and/or cancerous cells. U.S. patent application filed by Sengupta et al. having publication #20050266067 and entitled “Nanocell Drug Delivery System” is incorporated by reference herein for background on beneficial compositions deliverable by nano device technology. U.S. patent application filed by Kurzrock et al. having publication #20060067998 and entitled “Liposomal Curcumin for Treatment of Cancer” is incorporated by reference herein as related to cancerous cells and treatments therefor. U.S. patent application filed by Meininger having publication #20050053590 and entitled “Endothelium-Targeting Nanoparticle for Reversing Endothelial Dysfunction” discloses compositions beneficial in the treatment of endothelial cells damaged by diabetes, smoking, dyslipidemia, hypertension and cardiovascular disease.

It is another object of the invention to use the methods and apparatus disclosed herein with compressive technologies such as elastic wraps and hyperbaric chambers to protect surface tissue while increasing blood flow and oxygen concentration in treated tissues.

It is another object of the invention to use the methods and apparatus disclosed herein with compressive technologies such as elastic wraps and hyperbaric chambers to protect surface tissue while increasing blood flow and oxygen concentration in treated tissues.

It is a further object of the invention to provide a system and method that allows for deep penetration of decompressive energies in the form of vacuum forces into human or animal tissues.

### BRIEF DESCRIPTION OF THE DRAWINGS

FIG. 1 provides a detailed cut-away view of the dynamic load diffusion platform and seal of the present invention.

FIG. 2 provides a view of a portable lower extremity decompression device and chamber assembly.

FIG. 3 provides a view of a vessel for treating the hand and forearm areas.

FIG. 4 provides a view of a vessel for treating the hand and palm areas.

FIG. 5 provides a view of a vessel and apparatus for treating the hand and finger joints areas.

FIG. 6 provides an end view of the blood vessel response to proper decompressive treatment.

FIG. 7 provides a side view of the blood vessel response to proper decompressive treatment.

FIG. 8 provides an isometric view of the smart chip controlled vacuum charging system of the present invention.

FIG. 9 provides an isometric view of the dynamic load diffusion platform and seal mating assembly of the present invention.

FIG. 10 provides an isometric view of the interlocking pressurized load diffusion seal for an interlocking collar for easy on and off application.

FIG. 11 provides an isometric view of the treatment chamber for the entire lower half body necessary for deep artery treatment and enhancement of the present invention.

FIG. 12 is an illustration of the decompression gradient produced by application of decompression energy to tissue.

FIG. 13 is an isometric view of the dynamic energy and load action of one embodiment of the present invention.

FIG. 14 is an isometric view of the implantable decompression chamber embodiment of the present invention.

FIG. 15 is a graphic chart comparing dynamic load diffusion to static load distribution.

FIG. 16 is an isometric view of the double chambered decompression device of the present invention.

FIG. 17 is an isometric view of the dynamic fluid filled load diffusion seal of the present invention.

FIG. 18 illustrates the envisioned effects of Vascir™ decompression therapy on neurological tissues.

FIG. 19 presents an isometric view of the multi-valve release assembly.

FIG. 20 is an illustration of the effect of decompression energy applied to a cell.

FIG. 21 is an illustration of tissue having cancerous cells in combination with non-cancerous cells.

FIG. 22 is an illustration of the effect of decompression energy on cancerous cells as shown in FIG. 21.

FIG. 23 is an illustration of a cancerous cell rupturing under decompressive energy.

FIG. 24 is another embodiment of a decompression energy apparatus using vacuum for lower extremity tissue treatment.

FIG. 25 is another embodiment of a decompression energy apparatus using vacuum for tissue treatment for the hand and fingers.

### DETAILED DESCRIPTION—ELEMENT LISTING

| | |
|---|---|
| DYNAMIC FLUID FILLED LOAD DIFFUSION SEAL | 1 |
| VACUUM VESSEL | 2 |
| DYNAMIC ENERGY TRANSFER COLLAR | 3 |
| LONGITUDINAL ENERGY FLOW | 4 |
| DIAGONALLY UPWARD ENERGY FLOW | 5 |
| VERTICALLY UPWARD ENERGY FLOW | 6 |
| DYNAMIC ENERGY TRANSFER COLLAR DIRECTIONAL ANGLE | 7 |
| TRANSPARENT VACUUM VESSEL | 8 |
| BI-DIRECTIONAL CHECK VACUUM VALVE | 9 |
| PORTABLE LOWER EXTREMITY DECOMPRESSION DEVICE AND CHAMBER | 10 |
| ASSEMBLY | |
| FLUID FILLED CHAMBER | 11 |
| DYNAMIC ENERGY TRANSFER COLLAR MITTEN STYLE EMBODIMENT | 12 |
| PORTABLE UPPER EXTREMITY DECOMPRESSION DEVICE AND CHAMBER | 13 |
| ASSEMBLY | |
| DYNAMIC FLUID FILLED LOAD DIFFUSION SEAL MITTEN STYLE | 14 |
| EMBODIMENT | |
| THUMB DECOMPRESSION DEVICE AND CHAMBER ASSEMBLY | 15 |
| INDEX FINGER DECOMPRESSION DEVICE AND CHAMBER ASSEMBLY | 16 |
| MIDDLE FINGER DECOMPRESSION DEVICE AND CHAMBER ASSEMBLY | 17 |
| TRANSPARENT VACUUM VESSEL MITTEN STYLE EMBODIMENT | 18 |
| RING FINGER DECOMPRESSION DEVICE AND CHAMBER ASSEMBLY | 19 |
| PINKY FINGER DECOMPRESSION DEVICE AND CHAMBER ASSEMBLY | 20 |
| FINGER DEVICE ASSEMBLY MANIFOLD | 21 |
| SECONDARY TRANSPARENT VACUUM MULTIPLIER VESSEL | 22 |
| MOUNTING AND DIGIT SERVICING PLATFORM | 23 |
| VACUUM DELIVERY LINES | 24 |
| USER'S FOOT | 25 |
| USER'S LEG | 26 |
| OPPOSING SURFACE CONTACT AREA | 27 |
| CHAMBER MATING SECTION WITH THE DYNAMIC ENERGY TRANSFER | 28 |
| COLLAR | |
| TRANSPARENT VACUUM VESSEL INDIVIDUAL DIGIT STYLE ASSEMBLY | 29 |
| DISEASED UNTREATED BLOOD VESSEL OR ARTERIAL WALL THINNESS | 30 |
| (ANEURISM) | |
| DISEASED TREATED BLOOD VESSEL OR ARTERIAL WALL THINNESS | 31 |
| (ANEURISM) BEING STRENGTHENED | |
| DISEASED TREATED BLOOD VESSEL OR ARTERIAL WALL THINNESS | 32 |
| (ANEURISM) BEING PROGRESSIVELY STRENGTHENED | |
| DISEASED UNTREATED BLOOD VESSEL OR ARTERY WITH NARROWING | 33 |
| AND BRITTLENESS | |
| DISEASED BLOOD VESSEL OR ARTERY WITH EXPANSIONISM BEING | 34 |
| APPLIED AND TREATED | |
| DISEASED BLOOD VESSEL OR ARTERY BECOMING HEALTHY, | 35 |
| UNRESTRICTED AND FLEXIBILITY RESTORED | |
| BLOOD VESSEL WITH RESTRICTIVE BUILDUP AND BRITTLENESS ALONG | 36 |
| WITH WALL ABNORMALITY | |
| BLOOD VESSEL BREAK DOWN OF WALL BUILD UPS AND INCREASES IN | 37 |
| FLEXIBILITY AND STRENGTH | |
| UNRESTRICTED BLOOD FLOW AND INCREASED STRENGTH ALONG WITH | 38 |
| FLEXIBILITY | |
| DYNAMIC ACTION IMPLANTABLE DECOMPRESSION CHAMBER | 39 |
| NORMAL HEALTHY HUMAN CELL WITHOUT VACUUM DECOMPRESSION | 40 |
| APPLIED | |
| NORMAL HEALTHY HUMAN CELL WITH VACUUM DECOMPRESSION | 41 |
| BEING APPLIED | |
| NORMAL HEALTHY HUMAN CELL WITH VACUUM DECOMPRESSION | 42 |
| FULLY APPLIED WITH THIN MEMBRANE | |
| NORMAL HEALTHY HUMAN CELL AFTER TREATMENT IS OXYGEN | 43 |
| ENRICHED AND VIBRANT | |
| NORMAL HEALTHY HUMAN CELL AFTER TREATMENT WITH STRONGER | 44 |
| MEMBRANE WITH MORE FLEXIBILITY | |
| DISEASED HUMAN CELL WITHOUT VACUUM DECOMPRESSION APPLIED | 45 |
| DISEASED HUMAN CELL WITH VACUUM DECOMPRESSION BEING APPLIED | 46 |
| AND SEVERALLY STRESSED | |
| DISEASED HUMAN CELL WITH FULL VACUUM DECOMPRESSION APPLIED | 47 |
| RUPTURING MEMBRANE | |
| DISEASED HUMAN CELL WITH FULL VACUUM DECOMPRESSION APPLIED | 48 |
| RUPTURING NUCLEUS | |
| PORTABLE MITTEN STYLE DECOMPRESSION DEVICE AND CHAMBER | 49 |
| ASSEMBLY | |
| SMART CHIP CONTROLLED VACUUM CHARGING SYSTEM'S VACUUM PORT | 50 |
| SMART CHIP CONTROLLED VACUUM CHARGING SYSTEM DEVICE | 51 |
| ASSEMBLY | |
| SMART CHIP CONTROLLED VACUUM CHARGING SYSTEM'S ON BUTTON | 52 |
| SMART CHIP CONTROLLED VACUUM CHARGING SYSTEM'S DISPLAY | 53 |
| SCREEN | |
| SMART CHIP CONTROLLED VACUUM CHARGING SYSTEM'S LOCKING | 54 |
| SYSTEM | |
| SMART CHIP CONTROLLED VACUUM CHARGING SYSTEM'S ACCESS DOOR | 55 |
| TO SMART CHIP COMPARTMENT | |
| SMART CHIP CONTROLLED VACUUM CHARGING SYSTEM'S SMART CHIP | 56 |
| SMART CHIP CONTROLLED VACUUM CHARGING SYSTEM'S DC CONTACTS | 57 |
| SMART CHIP CONTROLLED VACUUM CHARGING SYSTEM'S DC BATTERY | 58 |
| POWER SOURCE | |
| SMART CHIP CONTROLLED VACUUM CHARGING SYSTEM'S EXTERNAL DC | 59 |
| SOURCE | |
| SMART CHIP CONTROLLED VACUUM CHARGING SYSTEM'S OFF BUTTON | 60 |
| MAIN COMPUTER SYSTEM FOR PROGRAMMING AND READING EXTERNAL | 61 |
| SMART CHIPS | |
| MAIN COMPUTER SYSTEM SMART CHIP INTERFACE DOCKING PORT | 62 |
| MAIN COMPUTER SYSTEM HUMAN INTERFACE AND INPUT DEVICE | 63 |
| DYNAMIC LOAD DIFFUSION PLATFORM AND SEAL MATING ASSEMBLY. | 64 |
| BLOOD VESSEL OR ARTERY WITH 0% VASCIR DECOMPRESSION APPLIED | 65 |
| BLOOD VESSEL OR ARTERY WITH 33% VASCIR DECOMPRESSION APPLIED | 66 |
| BLOOD VESSEL OR ARTERY WITH 100% VASCIR DECOMPRESSION APPLIED | 67 |
| BLOOD VESSEL OR ARTERY THAT IS NARROWED AND MOSTLY BLOCKED | 68 |
| BLOOD VESSEL OR ARTERY THAT IS WIDENED AND PARTIALLY BLOCKED | 69 |
| INTERLOCKING PRESSURIZED DYNAMIC FLUID FILLED LOAD DIFFUSION | 70 |
| SEAL RIGHT SIDE | |
| INTERLOCKING PRESSURIZED DYNAMIC FLUID FILLED LOAD DIFFUSION | 71 |
| SEAL LEFT SIDE | |
| INTERLOCKING PRESSURIZED DYNAMIC FLUID FILLED LOAD DIFFUSION | 72 |
| SEAL MALE MEMBER | |
| INTERLOCKING PRESSURIZED DYNAMIC FLUID FILLED LOAD DIFFUSION | 73 |
| SEAL FEMALE RECEIVER | |
| INTERLOCKING PRESSURIZED DYNAMIC FLUID FILLED LOAD DIFFUSION | 74 |
| SEAL MATING OF MALE FEMALE LEFT SIDE | |
| INTERLOCKING PRESSURIZED DYNAMIC FLUID FILLED LOAD DIFFUSION | 75 |
| SEAL MATING OF MALE FEMALE RIGHT SIDE | |
| INTERLOCKING PRESSURIZED DYNAMIC FLUID FILLED LOAD DIFFUSION | 76 |
| SEAL ASSEMBLY | |
| NERVE TISSUE DAMAGED AND WITHOUT VASCIR THERAPY | 77 |
| NERVE TISSUE DAMAGED AND WITH VASCIR THERAPY | 78 |
| BLOOD VESSEL OR ARTERY THAT IS FULLY EXPANDED AND HAS | 79 |
| MINIMUM BLOCKAGE | |
| HALF BODY DECOMPRESSION TREATMENT CHAMBER ASSEMBLY | 80 |
| DECOMPRESSION TREATMENT CHAMBER LOWER HALF OF CHAMBER | 81 |
| DECOMPRESSION TREATMENT CHAMBER TRANSPARENT UPPER HALF OF | 82 |
| CHAMBER | |
| DECOMPRESSION TREATMENT CHAMBER TOP SECTION OF FLEXIBLE | 83 |
| SHEET SEAL WITH ZIPPER | |
| DECOMPRESSION TREATMENT CHAMBER LOWER FLEXIBLE SHEET SEAL | 84 |
| WITH ZIPPER | |
| DECOMPRESSION TREATMENT CHAMBER LOWER FLEXIBLE SEAL ZIPPER | 85 |
| DECOMPRESSION TREATMENT CHAMBER LOWER SECTION OF FLEXIBLE | 86 |
| SEAL RUBBER-LIKE MATERIAL | |
| DECOMPRESSION TREATMENT CHAMBER UPPER FLEXIBLE SEAL ZIPPER | 87 |
| DECOMPRESSION TREATMENT CHAMBER LEFT LEG COMPARTMENT | 88 |
| DECOMPRESSION TREATMENT CHAMBER REMOVABLE LEG | 89 |
| COMPARTMENT DIVIDER | |
| DECOMPRESSION TREATMENT CHAMBER COMPRESSIBLE MATING SEAL | 90 |
| OF THE TOP AND LOWER CHAMBERS | |
| DECOMPRESSION TREATMENT CHAMBER RIGHT LEG COMPARTMENT | 91 |
| DECOMPRESSION TREATMENT CHAMBER PADDED RESTING AREA FOR | 92 |
| THE BUTTOCKS | |
| OPPOSING FORCES RIGHT SIDE | 93 |
| OPPOSING FORCES LEFT SIDE | 94 |
| DYNAMIC VERTICAL ENERGY | 95 |
| DYNAMIC EQUALIZATION OF ENERGY INSTANTANEOUSLY THROUGH | 96 |
| THE FLUID FILLED SEAL VESSEL CONTACT POINT | |
| DYNAMIC EQUALIZATION OF ENERGY INSTANTANEOUSLY THROUGH | 97 |
| THE FLUID FILLED SEAL VESSEL CONTACT POINT | |
| VACUUM TUBING WITH PRESSURE SENSITIVITY FOR SAFETY | 98 |
| OPEN | 99 |
| OPEN | 100 |
| VACUUM PRESSURE SENSOR | 101 |
| OXYGEN (O2) SENSOR | 102 |
| WATER H2O SENSOR | 103 |
| THERMAL SENSOR | 104 |
| COLLARS VESSEL MATING GROVE | 105 |
| BLOOD VESSEL OR ARTERY | 106 |
| LEAK PROOF SEAM AND JOINT | 107 |
| DYNAMIC LIVE LOADS DIAGONALLY | 108 |
| DYNAMIC LIVE LOADS VERTICALLY | 109 |
| DYNAMIC LIVE LOADS LONGITUDINAL | 110 |
| DOWNWARD ENERGY FLOW | 111 |
| DIAGONALLY DOWNWARDLY ENERGY FLOW | 112 |
| OPEN | 113 |
| EXTERNAL TRANSPARENT VACUUM VESSEL | 114 |
| INTERNAL TRANSPARENT VACUUM VESSEL | 115 |
| BI-DIRECTIONAL CHECK VACUUM VALVE FOR INTERNAL CHAMBER | 116 |
| DOUBLE CHAMBERED DECOMPRESSION DEVICE ASSEMBLY | 117 |
| MULTI VALVE O-RING | 118 |
| MULTI VALVE BUTTON SHEATH WITH CUT OUT | 119 |
| MULTI VALVE ACTIVATION BUTTON WITH BLEED | 120 |
| MULTI VALVE ACTIVATION BUTTON BLEED OFF CHANNEL | 121 |
| MULTI VALVE CHECK VALVE DIAPHRAM | 122 |
| MULTI VALVE CHECK VALVE HOUSING | 123 |
| MULTI VALVE DUAL PURPOSE INLET EXHAUST PORTS | 124 |
| MULTI VALVE RETURN SPRING | 125 |
| MULTI VALVE RETURN SPRING RETAINER WITH EVACUATION NUBS | 126 |
| MULTI VALVE STOP PLATE | 127 |
| MULTI VALVE RETURN SPRING RETAINER EVACUATION PORT | 128 |
| MULTI VALVE THREADED END | 129 |
| MULTI VALVE ASSEMBLY | 130 |
| VACUUM TUBING WITH PRESSURE SENSITIVITY FOR SAFETY | 131 |
| ATMOSPHEREIC PRESSURE, DECOMPRESSSIVE ENERGY IS NEUTRAL | 132 |
| DECOMPRESSIVE ENERGY UNITS (DEU) | 133 |
| (DE) DECOMPRESSIVE ENERGY | 134 |
| SOFT AND/OR PERMEABLE TISSUE | 135 |
| VESSEL CONTAINING DECOMPRESSIVE ENERGY | 136 |
| DECOMPRESSIVE ENERGY GRADIENT | 137 |
| OPEN | 138 |
| OPEN | 139 |
| CANCER CELLS IN OTHERWISE HEALTHY HUMAN WITHOUT VACUUM | 140 |
| DECOMPRESSION APPLIED | |
| CANCER CELL IN OTHERWISE HEALTHY HUMAN WITH VACUUM | 141 |
| DECOMPRESSION BEING APPLIED | |
| OPEN | 142 |
| MEMBRANE RUPTURE OF CANCER CELL IN OTHERWISE HEALTHY HUMAN | 143 |
| WITH VACUUM DECOMPRESSION BEING APPLIED | |

### DETAILED DESCRIPTION

In present application, the following preceding terms are defined accordingly: A cell is defined as the individual unit that makes up all of the tissues of the body. All living things are made up of one or more cells. Tissue is defined as a group of similar cells from an animal or mammal united to perform a specific function. Soft tissue is defined as tissue that is not bone. As defined herein, tissue or soft tissue may include organs. Vacuum is defined as the condition of rarefaction, or reduction of pressure below that of the atmosphere, in a vessel, tissue or a cell. This action of creating a vacuum creates a state of energy exchange in what is known as decompressive energy. A state of stable vacuum contains potential decompressive energy. That potential is released, generated, delivered and/or manufactured when it acts on or interacts with other matter in its realm of influence and interaction. Cancer is a term for diseases in which abnormal cells divide (mitosis) without control. Cancer cells can invade nearby tissues and spread through the bloodstream and lymphatic system to other parts of the body (metastasis). Cancer cells also avoid natural cell death (apoptosis). The vascular system is defined as the cardiovascular and lymphatic systems collectively, of a mammal or animal; also referred to as the circulatory system. Pharmacological is a therapy regimen that relies on drugs or includes drugs.

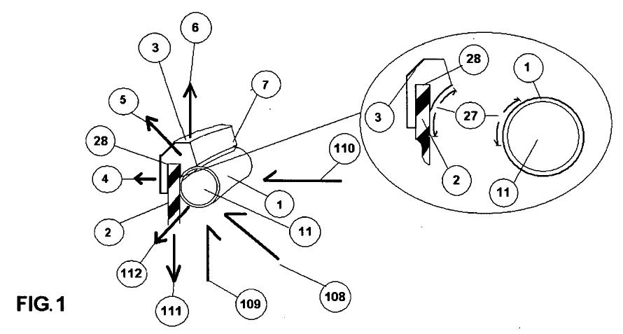

Referring now to the drawings, wherein like reference numerals designate identical or corresponding parts throughout the several views, FIG. 1 is a schematic view of the dynamic load diffusion platform and seal of the present art.

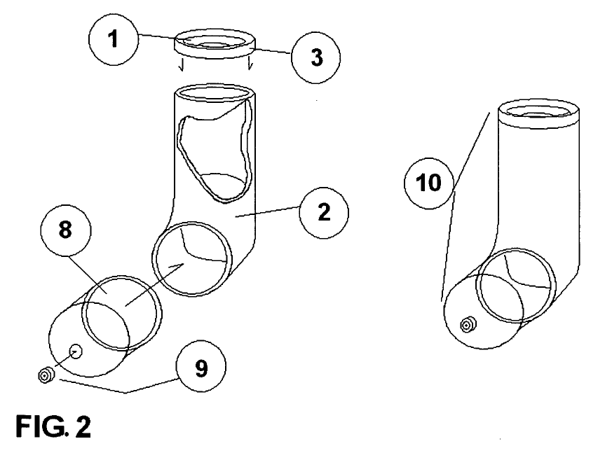

As shown by FIG. 1, the present invention functions to allow a safe interface with living tissue while allowing application of dynamic energies to living tissues through application of vacuum. The method and apparatus of the present invention as disclosed herein allows energy to be, but not limited to being, absorbed, burned up, utilized, transferred, redirected, divided, equalized, balanced, stabilized, minimized, transformed, disseminated, channeled, combined, limited, misdirected, vectored, controlled as in the directional flow of stress forces. The present invention also allows for changes in individual characteristics of the load energy being developed between the contact areas of the living tissue and the various forms of decompression chambers or vessels as shown in the following FIGS. 2, 3, 4, 5, 11, 13, 14 and 16.

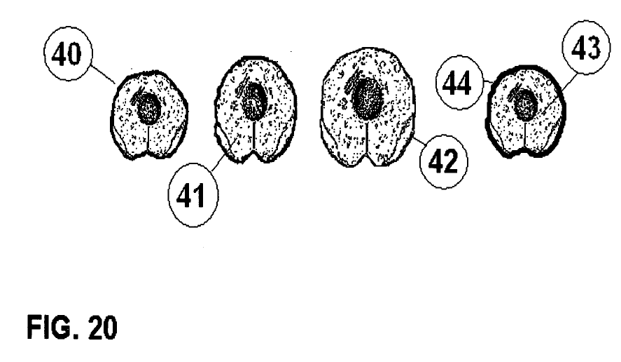

In the presence of vacuum and decompression, living tissues reacts in an exponential manner to vacuum energy, thus expanding and stressing the very biological structures that hold the cells and cellular matter together, as illustrated by FIG. 20. During the decompression process, the normal cell 40 and its membrane are temporarily expanded, stretched and thinned, 41 and 42, by the process of deep penetrating decompression due to and dependent upon the amount of vacuum in the particular chamber being used as shown in FIGS. 2, 3, 4, 5, 11, 12, 14 and 16. Because the amount of dynamic energy present and delivered by the chambers shown in FIGS. 2, 3, 4, 5, 11, 12, 13, 14 and 16 is directly related to the shape, design and volume of the vessel's chambers, the amount of penetrating energy being delivered to the tissue is thus directly related to these same values.

It is the stress load placed on the tissue that stimulates certain immune system, as well a given biological responses, not only at a cellular level but also at the atomic levels. During and after this process there appears to be a communication of some type, probably electromagnetic, that senses the change and acts in a way to communicate between the biological systems within the body itself, to indicate and/or direct a growth and/or repair response. During this application process the increase in blood flow, its nutrients and oxygen levels have been shown to increase in excess of three-hundred percent (300%). The tissue at the cellular level responds to the increased blood flow, stress and communication processes to determine a proper response, i.e. growth, repair, strengthening, increase in flexibility, regeneration, histogenesis and so on. There is no single response which does not also involve some stimulation of the other responses. Thus, in order for the deep penetration of decompressive energy to properly expand tissue, its force has to be directed, accelerated, controlled and regulated. The dynamic nature of vacuum applied decompressive energy and its consequential outcome of developing stresses and loads associated with this type of dynamic action and interaction thus creates a healthy cellular response 44 in healthy tissues and cells 40. It is the objective of each element, design and embodiment as set forth in this application to support this combination of responses.

The design criteria of this invention is to include, but is not be limited to, the dynamic action of decompression on living tissues which is well documented, as is also the fragileness and delicateness of living tissues. It is also well known that the brittleness and harshness of crushing forces, along with elastic and inelastic stress, linear and non-linear stresses, live and dead loads, tear, shear and all things of the physical universe are made up of energy. This energy is given specific names due to their individual characteristics of travel, transfer and exchange. This advanced design takes into consideration the dynamic nature of energy and can virtually and instantly handle and transfer energies action upon and in connection with the dynamic load diffusion seal.

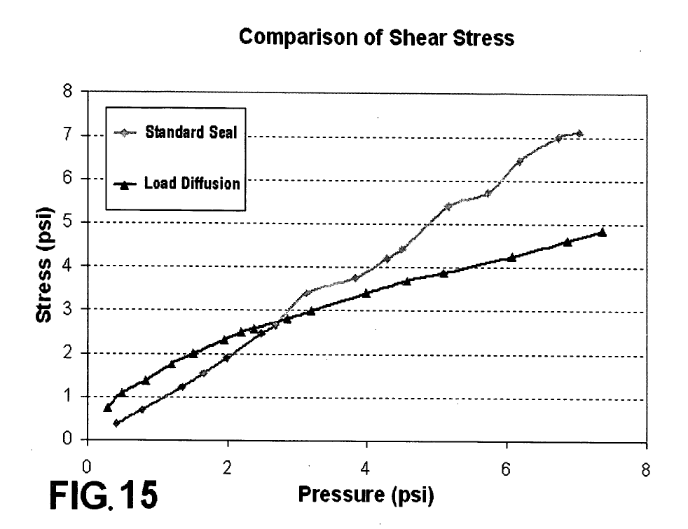

Computer modeling indicating the significant difference between load distribution and load diffusion has previously been completed as illustrated by the results found at FIG. 15. Load diffusion allows for the virtually instantaneous and dynamic response to a plurality of static, dynamic and radial type energy forces at one time.

In load distribution, stress concentrations arise from any abrupt change in the geometry of a specimen under loading. As a result, the stress distribution is not uniform throughout a cross section. See Astronautic Structures Manual (ω-Line), NASA MSFC (Marshall Space Flight. Center), 1975. The load diffusion platform and seal of the present invention eliminates stress concentrations by a virtual instantaneous balancing act that combines the actions of fluidic energy transfer and static load transfer with the mechanical action of compressibility, elasticity and static distributions.

This is accomplished as indicated in FIG. 1 by the combination of the five elements shown therein including the chamber mating section with dynamic energy transfer collar 3, the opposing surface contact area 27, elastic load diffusion seal 1, fluid filled chamber 11 and the vacuum vessel 2 wall. These elements work together to provide the necessary productivity and synergy required to produce the load diffusion for application to treatment of tissues for various ailments such as cancer, peripheral vascular disease and other circulatory diseases.

As shown in FIG. 1, dynamic live loads diagonally 108, dynamic live loads vertically 109 and dynamic live loads longitudinal 110 (hereinafter load energy vectors) are applied to dynamic fluid filled load diffusion seal 1 (hereinafter load diffusion seal) which absorbs burns up, utilizes, transfers, redirects, divides, equalizes, balances, stabilizes, minimizes, transforms, disseminates, channels, combines, limits, misdirects, vectorizes, meters, compensates, and changes directional flow of stress forces and even changes individual characteristics of the load energy. Once the load diffusion seal 1 is loaded with stress energy, it then directs the energy instantaneously to the mating area known as the opposing surface contact area 27. The opposing surface contact area 27 is a critical part of the pathway to load diffusion, as it acts as footed flanges. To be generally optimal, it should be at least one-fourth the diameter of the load diffusion seal 1 and placed at the lateral centerline of said periphery to incoming load energy vectors 108, 109 and 110. Though less or more than one-quarter opposing surface contact area 27 “arcuate configuration” may be used, it is thought that the one-quarter measurement requirement is a good median line for optimal energy transfer.

The opposing surface contact area 27 must be partially and/or wholly positively bonded to load diffusion seal 1 as to allow for the best possible energy transfer pathway and have an arcuate configuration that matches the arc of the load diffusion seal's 1 surface.

To maximize the energy transfer placed upon load diffusion seal 1 by load energy vectors 108, 109 and 110 it is critical that they are transmitted in a fluid-like action, thus the load diffusion seal 1 must be fluid filled. This fluid-like action is not unlike water or air, always seeking equilibrium automatically. This auto response or equilibrium seeking attribute reduces the shear, tear, and crushing forces applied to tissues while continuously maintaining an airtight seal with the supporting or surrounding tissues. Therefore, this seal design is synergistic in that the greater the vacuum inside the vacuum vessel 2, the more positive the sealing capabilities of the load diffusion seal 1.

The load forces or load energy vectors represented by 108, 109 and 110 are transmitted in a fluid-like action and directed and distributed in such a manner and in somewhat a radial direction. FIG. 1 illustrates that the load energy vectors 108, 109 and 110 are reduced in amount as the load diffusion seal 1 is compressed. This reduction in force or energy is due to the resistance to compression provided by the elasticity of the seal material and its inner fluid filled center. This dual action resists compression, thus utilizing or dissipating or diffusing even more of the energy or force being delivered by load energy vectors 108, 109 and 110.

This energy, once it has sufficiently loaded the load diffusion seal 1 and directed through to the opposing surface contact area 27, is now delivered and directed to the fixed and solid materials that make up the rest of the device. This energy is distributed in a radial type lateral action as shown as illustrated in FIG. 1 at elements 4, 5, 6, 111 and 112. Due to the nature of the design, the loaded energy then follows the direction of least resistance and follows the pathway to the vacuum vessels 2 wall for distribution of the energy to the rest of the device and away from the supporting tissues of the user. Each time the energy meets resistance, a portion of energy is utilized or dissipated (or diffused), thus reducing the total amount of energy being transmitted. It is important to note that this happens in a fluid and continuous manners and works with a synergy that is unique to this invention.

The mating for the vacuum vessel 2 wall and the dynamic energy transfer collar 3 is accomplished by the positively affixed chamber mating section with the dynamic energy transfer collar 28. This mating provides for a continuous and dynamic energy flow through it, as if it were a solid and homogenous material, and also automatically acts like an extension to the surface contact area 27 of the load diffusion seals 1 mating with the dynamic energy transfer collar 3. The significance of this feature is to increase the contact area while under loads. Element 7 in FIG. 1 is a reduction in mass of the dynamic energy transfer collar 3 and allows a smooth finished transition on the top side of dynamic energy transfer collar 3.

FIG. 2 provides a view of a portable lower extremity decompression device and chamber assembly 10. This embodiment of the device is a portable lower extremity decompression device and chamber assembly 10 which is made up of a vacuum vessel 2 designed in such a way as to allow the human foot to fit comfortably inside and at the same time to be strong enough to handle a large amount of vacuum pressure without imploding. The visual inspection section of the portable lower extremity decompression device and chamber assembly 10 (also referred to as assembly) is the transparent vacuum vessel 8, which allows the doctor or user to view the area that is being treated. The transparent vacuum vessel 8 is topped with a bi-directional check vacuum valve 9 used to evacuate the atmosphere inside the vacuum vessel 2 or assembly 10. On top of the portable lower extremity decompression device and chamber assembly 10 is the human or user interface element called the dynamic energy transfer collar 3, which contains the load diffusion seal 1. This unit is designed to provide a non-invasive way to stimulate the vascular and neurological functions and enhance oxygenation of the extremity being treated by the portable lower extremity decompression device and chamber assembly 10.

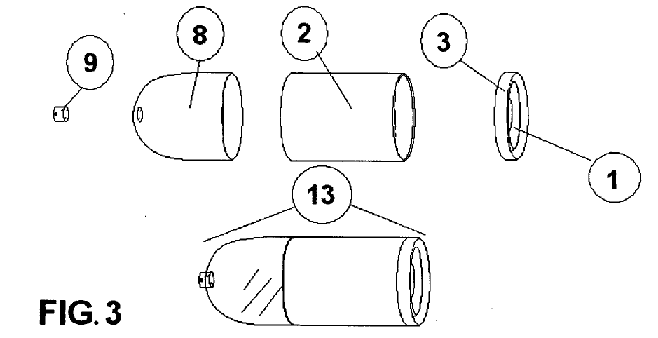

FIG. 3 is a view of vessel for treating the hand and forearm areas. This device embodiment is of a portable upper extremity decompression device and chamber assembly 13 which is made up of a vacuum vessel 2 designed in such a way as to allow the arm and hand to fit comfortably inside and at the same time to be strong enough to handle a large amount of vacuum pressure without imploding.

The visual inspection section of the portable upper extremity decompression device and chamber assembly 13 is the transparent vacuum vessel 8, which allows the doctor or user to view the area that is being treated. The transparent vacuum vessel 8 is topped with a bi-directional check vacuum valve 9 used to evacuate the atmosphere inside the vacuum vessel 2 or portable upper extremity decompression device and chamber assembly 13. On top of the portable upper extremity decompression device and chamber assembly 13 is the human or user interface element called the dynamic energy transfer collar 3, which contains the load diffusion seal 1. This unit is designed to provide a non-invasive way to stimulate the vascular and neurological functions and enhance oxygenation of the extremity being treated by the portable upper extremity decompression device and chamber assembly 13.

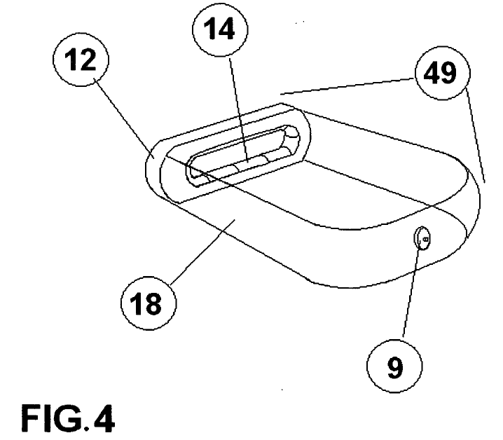

FIG. 4 provides a view of a vessel for treating the hand and palm areas. This device embodiment is of a portable mitten style decompression device and chamber assembly 49 which is made up of a transparent vacuum vessel mitten style embodiment 18 designed in such a way as to allow the palm of the hand and fingers only to fit comfortably inside the vessel and at the same time to be strong enough to handle a large amount of vacuum pressure without imploding. The transparent vacuum vessel mitten style embodiment 18 which allows the doctor or user to view the area that is being treated and is topped with a bi-directional check vacuum valve 9 used to evacuate the atmosphere inside the portable mitten style decompression device and chamber assembly 49. The human or user interface element of portable mitten style decompression device and chamber assembly 49 is called the dynamic energy transfer collar mitten style embodiment 12, which contains the load diffusion seal 14. This unit is designed to provide a non-invasive way to stimulate the vascular and neurological functions and enhance oxygenation of the extremity being treated.

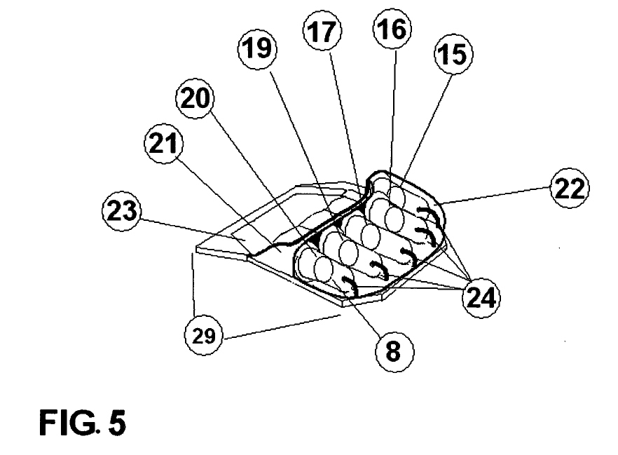

FIG. 5 is a view of a vessel and apparatus for treating the hand and figure joints areas. This device embodiment is of a transparent vacuum vessel individual digit style assembly 29, which is made up of many individual interconnected vacuum vessels 2. Each vacuum vessel 2 is designed for the appropriate digit. The elements include the thumb decompression device and chamber assembly 15, the index finger decompression device and chamber assembly 16, middle finger decompression device and chamber assembly 17, ring finger decompression device and chamber assembly 19 and the pinky finger decompression device and chamber assembly 20. Each individual assembly together form the total device embodiment known as the transparent vacuum vessel individual digit style assembly 29 and can be configured with or without all the individual devices installed. Each assembly contains the basic units of assembly just like the rest of the embodiments of this invention. The transparent vacuum vessel individual digit style assembly 29 has a resting plate for the palm of the hand called the mounting and digit servicing platform 23 where all the individual digit assemblies are mounted via the finger device assembly manifold 21, which as a option can have applied to the secondary transparent vacuum multiplier vessel 22 to increase the reactive state of decompression.

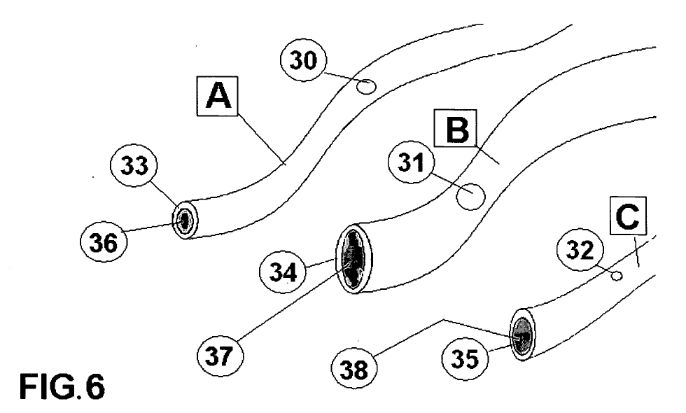

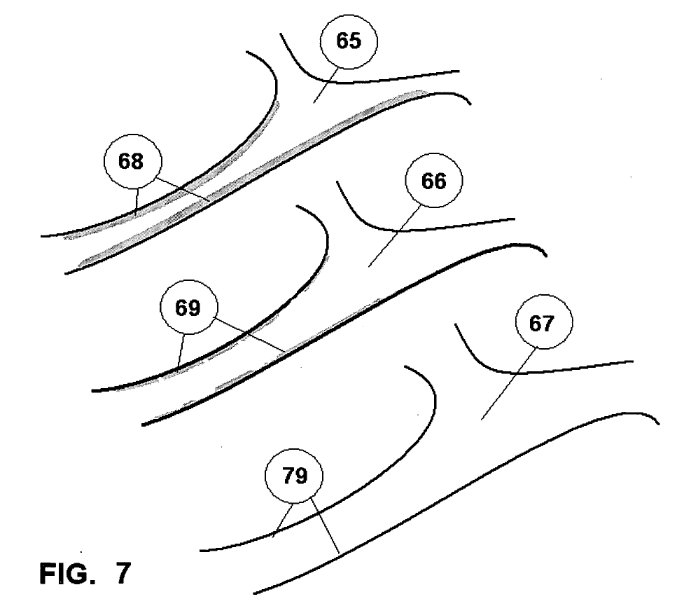

FIGS. 6 and 7 illustrate the intended vascular development under Vascir™ Decompressive Therapy. FIG. 6 provides a view of the blood vessel response to proper treatment. As illustrated in FIG. 6, there are three (3) stages of vascular tissue (blood vessel). Illustration A of FIG. 6 represents an average diseased untreated blood vessel or artery with narrowing and brittleness 33. Treatment with decompression energy can and does affect the value of health and effectiveness of said vessel. Illustration B represents the same diseased untreated blood vessel or artery with narrowing and brittleness 33 and depicts how the diseased blood vessel or artery expands under decompression. Note the blood vessel breakdown of wall and buildups and increases in flexibility and strength 37. This type of expansion is known to cause the vessel to become more flexible, elastic and stronger as shown in illustration B wherein the diseased blood vessel or artery is becoming healthy, unrestricted and flexibility restored (shown at element 37). After the proper treatment regime with decompressive forces, the artery becomes unrestricted, blood flow capacity is increased, and strength along with flexibility are also enhanced as illustrated in C at unrestricted blood flow and increased strength along with flexibility 38. It is well known from the study of blood vessel aneurisms, that the weakness in the vessel wall will repair itself over time if the vessel does not first rupture. The theory presented is that the vessel senses or responds at the thinned or the stressed area and begins to repair itself or auto-generate tissue. It is believed that this invention enhances the body's natural repairing mechanism. Illustration A shows a diseased untreated blood vessel with arterial wall thinness 30 similar to the condition that may be found with a potential aneurism 30 vessel situation.

During decompressive therapy, the gentle expansion or stress upon the vessel can stimulate the vessel to heal itself quicker and help to increase the cell wall thickness through auto-generation of tissue. Furthermore, the slow expansion and contraction of the vessel walls is believed to allow the vessel to become more supple and, thus stronger. This results from the combination of both tissue growth stimulation and the breakdown of vessel wall build-up. Plaque or vessel wall build-up is known to be inelastic or brittle, thus it cannot adhere to the vessel wall as the vessel wall expands.

Illustration B depicts how this might happen, with the diseased treated blood vessel or arterial wall thinness (aneurism) being strengthened 31; diseased treated blood vessel or arterial wall thinness (aneurism) being strengthened 31; diseased treated blood vessel or arterial wall thinness (aneurism) being progressively strengthened 32; diseased untreated blood vessel or artery with narrowing and brittleness 33; diseased blood vessel or artery with expansionism being applied and treated 34; diseased blood vessel or artery becoming healthy, unrestricted and flexibility restored 35; and, Blood vessel with restrictive buildup and brittleness along with wall abnormality 36. Additionally, Illustration B shows blood vessel breakdown of wall build-ups or scale and increases in flexibility and strength 37. Illustration C shows unrestricted blood flow and increased strength along with flexibility 38.

FIG. 7 illustrates a sectional side view of the blood vessel or artery in various stages of treatment. In the illustration found at FIG. 7, a sectional side view of the blood vessel or artery with 0% Vascir™ Decompression Therapy applied 65 is shown. As shown in this illustration, the blood vessel or artery is narrowed and mostly blocked 68. The middle illustration shows that blood vessel or artery with 33% Vascir™ Decompression Therapy applied is expanded or widened and now a blood vessel or artery that is widened and partially blocked 69. Finally, the bottom illustration shows a blood vessel or artery with 100% Vascir™ Decompression Therapy applied 67 is now a blood vessel or artery fully expanded and has minimum blockage 79, thereby improving and enhancing the condition of the tissue at both the micro-vascular and macro-vascular levels through improved artery or vessel condition, improved blood flow, and both cellular response and overall improved body health allowed by the preceding results. The apparatus may also be used with a pharmacological composition beneficial to increasing vascular elasticity and permeability by introducing the pharmacological compositions into said tissue in combination with the vacuum forces. This beneficial use of decompressive energy on the tissue to stimulate increased blood flow and cellular response may be further improved upon by introducing a pharmacological composition beneficial for healthy vascular angiogenesis and vasculogenesis into the tissue in combination with said decompressive energy. Examples of such pharmacological compositions include, but not limited to those named, generally including the class of drugs known as peripheral vasodilators, anticoagulants, beta blockers, combined alpha and beta blockers, central alpha agonists, peripheral alpha-1 blockers, angiotensin converting enzyme (ACE) inhibitors, calcium channel blockers and fenoldopam and combinations thereof. Use and delivery of these pharmacological compositions may be further improved if used in combination with nano-devices, including nano-shells and nanocells, for improved delivery and targeting of the pharmacological compositions to the treated tissues.

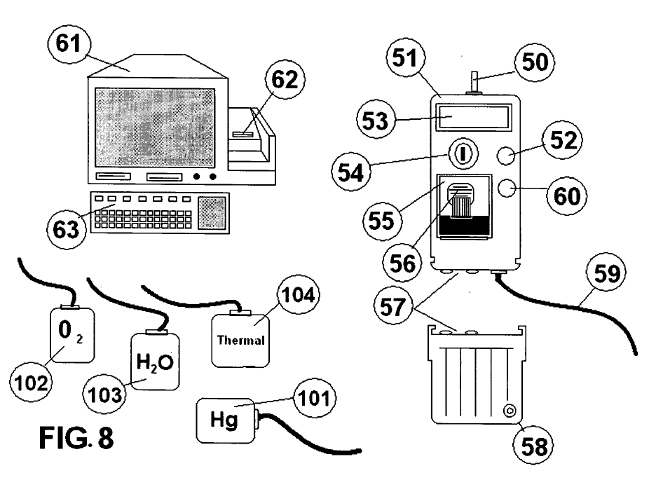

FIG. 8 provides an isometric view of the smart chip controlled vacuum charging system of the present invention. The smart chip system disclosed herein allows for automated operation and patient or user diagnosis. The smart chip and system disclosed herein for this device and devices are designed so that the attending physician can monitor the use of the Vascir™ Decompressive Therapy when the device is used both inside and outside the clinical setting. The smart chip controlled vacuum charging system's smart chip 56 (hereinafter smart chip) will be programmed by the physician's office via the main computer system for programming and reading external smart chips 61 (hereinafter main computer system), which in turn cannot only be used for medical diagnoses but can also be programmed through the smart ship 56 in combination with main computer system smart chip interface docking port 62 (hereinafter docking port) for downloading the physician's prescription and/or protocols. Once the smart chip 56 is programmed in docking port 62, it is then removed by a staff member in the physician's office and placed into the smart chip controlled vacuum charging system device assembly 51. This will allow it to operate virtually autonomously as prescribed and programmed by said physician through main computer system human interface and input device 63 and the main computer system 61 for programming and reading of the smart chip 56.

The smart chip 56 has the ability to store not only the instructions for operation prescribed by the physician, but also records and controls the treatment device on/off and date, length of running time, amount of pressures used and solid or pulsated application of Vascir™ decompression energy. The smart chip 56 in combination with various sensors found on the device will record the oxygen levels via the oxygen sensor 102, temperatures or thermal readings via the thermal sensor 104, atmospheric water vapor content via the water sensor 103 and even monitor the inches and/or millimeters of vacuum pressure (Hg) via the vacuum pressure sensor 101 before, during and after application. This collection information will be stored to the storage area for download to the physician's main unit in the office or clinic on the users/patients next visit.

The smart chip controlled vacuum charging system device assembly 51 consists of many different controls for human interfacing and operation. For example, the smart chip controlled vacuum charging system's vacuum port 50 is the delivery port for the bi-directional check vacuum valve 9. The smart chip controlled vacuum charging system device assembly 51 has several ways it can communicate with the user. The digital display is for visual communication between device and operator is the smart chip controlled vacuum charging system's display screen 53. An audio tone and/or tones and flashing lighted array all are used to communicate with the operator/patient. For security, the smart chip 56 utilizes a smart chip controlled vacuum charging system's locking system 54, to protect it from patient interference. The smart chip 56 may only be removed with a proper key used to unlock the chip from the smart chip controlled vacuum charging system device assembly 51 via the smart chip controlled vacuum charging system's access door to smart chip compartment 55.

The smart chip controlled vacuum charging system device assembly 51 is a portable device and can be power from a smart chip controlled vacuum charging system's external direct current (DC) source 59 or with its own smart chip controlled vacuum charging system's DC battery power source 58, which supplies power though the smart chip controlled vacuum charging system's DC contacts 57. Mounted on the exterior of said smart chip controlled vacuum charging system device assembly 51 is a patient controlled smart chip controlled vacuum charging systems off button 60 for quick and easy termination of operation any time the patient so deems it for safety and security.

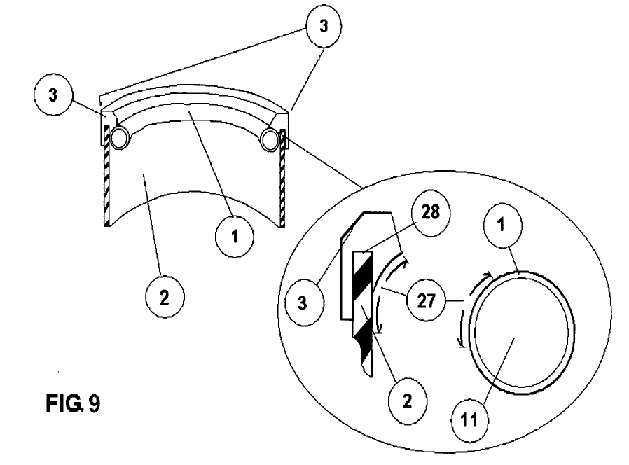

FIG. 9 provides an isometric view of the dynamic load diffusion platform and seal mating assembly 64 of the present invention. This figure illustrates that to work properly, the dynamic energy transfer collar 3 must be positively affixed to the vacuum vessel 2 wall via the chamber mating section with the dynamic energy transfer collar 28 and that the load diffusion seal 1 with its fluid filled chamber 11 must be positively affixed to the opposing surface contact area 27 of the dynamic energy transfer collar 3. As shown, the combination of elements work together to create an airtight seal.

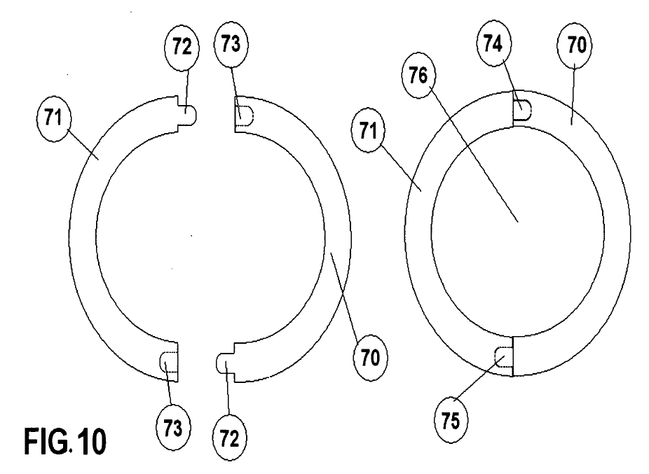

FIG. 10 provides an isometric view of the interlocking pressurized load diffusion seal for an interlocking collar for easy on and off application. Interlocking pressurized dynamic fluid filled load diffusion seal right side 70 and left side 71, respectively, are designed to allow for multiple segments and/or parts to fit together in such a way as to create an effective vacuum seal at each coupling area or joint while allowing multiple segments to be used together for “size” to the user. Each interlocking pressurized dynamic fluid filled load diffusion seal male member 72 and interlocking pressurized dynamic fluid filled load diffusion seal female receiver 73 will allow for a fitting that creates an air and vacuum tight fit as illustrated with interlocking pressurized dynamic fluid filled load diffusion seal mating of male female left side 74 and right side 75, respectively. The fitted ends may be made in such a way as to still allow for maximum compressibility and flexibility of the interlocking pressurized dynamic fluid filled load diffusion seal assembly 76. The fluidity or communication of fluid-like properties is maintained via the interlocking seal to allow the combination of desired functional feature of sizing and the required load diffusion seal properties for inherent dynamic reaction and diffusion of the dynamic energies and forces applied.

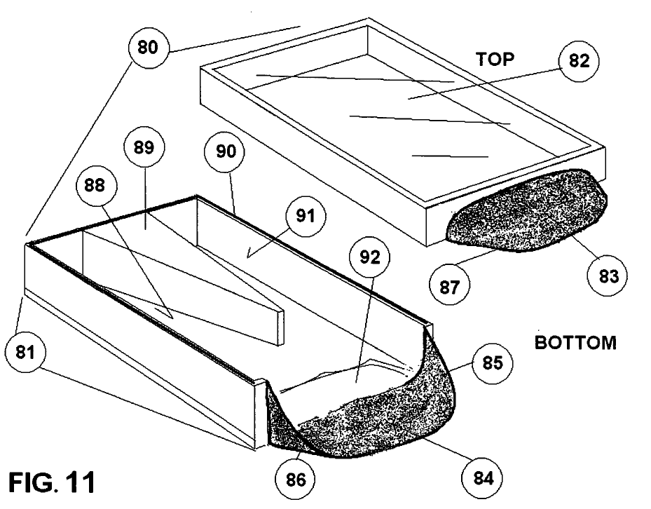

FIG. 11 provides an isometric view of the treatment chamber for the entire lower half body necessary for deep artery treatment and enhancement via the present invention. Half-body decompression treatment chamber assembly 80 is designed to stimulate and enhance the tissue of the lower half of the human body through decompressive energy. This embodiment allows dynamic vacuum energy to penetrate the body and cause the expansion of the tissues to enhance function and application of the body's natural biological systems. The main components of the half-body decompression treatment chamber assembly 80 are the decompress treatment chamber lower half of chamber 81, decompression treatment chamber transparent upper half of chamber 82, decompression treatment top section of flexible sheet seal with zipper 83, decompression treatment chamber left leg compartment 88, decompression treatment chamber removable leg compartment divider 89, decompression treatment chamber right leg compartment 91 and decompression treatment chamber compressible mating seal of the top and lower chambers 90. These components come together to produce a vacuum tight assembly that has an open end that has attached to it flexible sheets that can wrap around the patient placed in the box and be zipped tight to form a vacuum-tight seal against the user of the device. The decompression treatment chamber top section of flexible sheet seal with zipper 83 is mated to decompression treatment chamber lower flexible sheet seal 84 with zipper, decompression treatment chamber lower flexible seal zipper 85, decompression treatment chamber lower section of flexible seal rubber-like material 86 and decompression treatment chamber upper flexible seal zipper 87. Embedded in the decompression treatment chamber lower half of chamber 81 is a decompression treatment chamber padded resting area for the buttocks 92. Although not shown, the present invention may also be used in combination with hyperbaric oxygen patient treatment, such as taught by U.S. Pat. No. 6,484,716, which is incorporated by reference herein. A patient placed in the embodiment of FIG. 11 may also separately or concurrently be subjected to hyperbaric oxygen treatment effectively loading the cardiovascular system with increased levels of oxygen via the respiratory system for improved delivery through tissue having reduced vascular capacity.

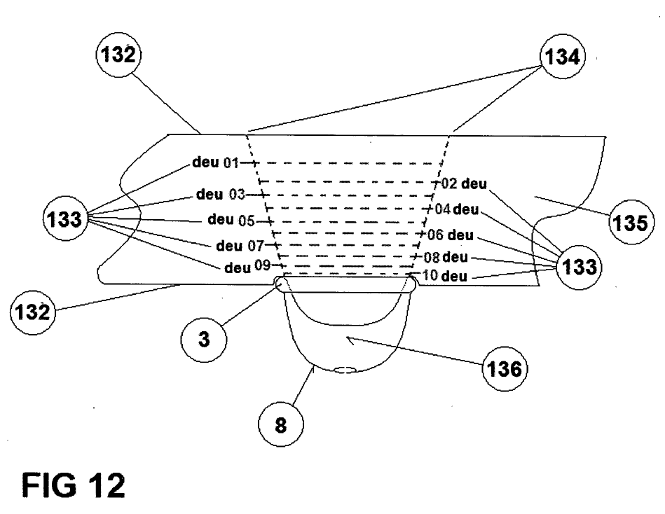

FIG. 12 is a graphical view of the decompressive energy gradient 137. It attempts to represent the pattern of deliverable decompressive energy to the soft and/or permeable tissue 135 being treated. The energy being greatest on the application surface of the soft and/or permeable tissue 135 and diminishing as it deeply penetrates the depths of the soft and/or permeable tissue 135. The units of measure are referred to herein as DEU 133 (decompressive energy unit). This diagram of DEU 133 and decompressive energy gradient 137 is a generalized visual representation of a snap shot in time. The decompressive energy gradient 137 is a dynamic event and the DEUs 133 will change depending on soft and/or permeable tissue 135 density, the level of decompressive energy in the vessel containing decompressive energy 136, application time and a plurality of additional factors.

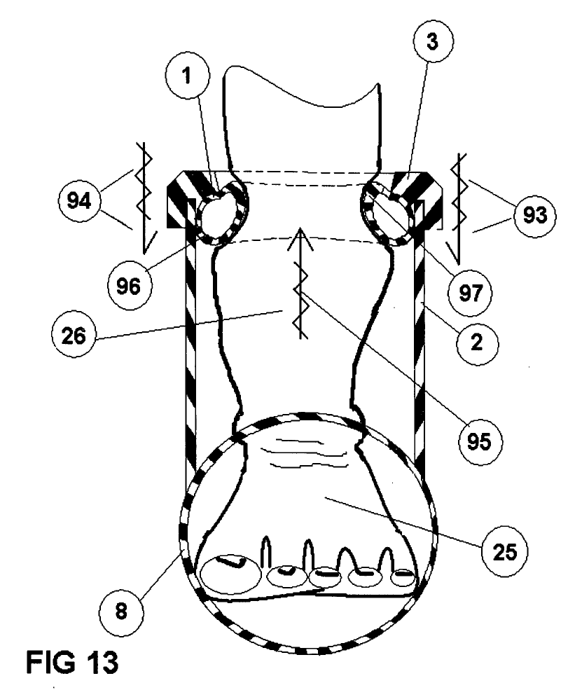

FIG. 13 is an isometric view of the dynamic energy and load action of the present invention. As shown, the user's foot 25 has been placed inside the circulatory and neurological enhancement device for decompressive therapy. The user's foot 25 rests on the bottom of transparent vacuum vessel 8. At the top of the transparent vacuum vessel 8 has been placed the dynamic energy transfer collar 3, which is engaged with the load diffusion seal 1. The load diffusion seal 1 in combination with the dynamic energy transfer collar 3 are designed to “fit” around an upper portion of the user's leg 26 and provide dynamic equalization of energy instantaneously through the fluid filled seal vessel contact points 96 and 97 upon application of the vacuum. At no time does the “hard” collar or vessel wall contact the user's leg 26 or the user's foot 25. As can be visualized from this figure, as vacuum energy is applied through decompression, the right and left sides of the interior of vessel opposing forces, represented by opposing forces right side 93 and opposing forces left side 94 respectively, dynamically equalize in response to the application of vacuum and decompression of the interior of vessel that results in an upper ward pull of the transparent vacuum vessel 8 as represented by dynamic vertical energy 95. Repeated treatment of tissues, such as with the lower extremity unit shown at FIG. 13, will increase the health of the tissues and the vascular system of the tissue as indicated by vascular elasticity, vascular strength, vascular blood flow rates, tissue genesis, vascular density and/or vascular permeability. Although not shown, the various embodiments of the present invention may also be used in combination with compressive means such as a compressive wrap comprised of elastic material to reduce edema, increase patient comfort, reduce discoloring of tissue and help facilitate achievement of greater vacuum pressures. Other variations such as those taught in U.S. Pat. Nos. 6,893,409; 6,488,643; and 6,135,116, and incorporated by reference herein, and may be used as those skilled in the arts will appreciate.

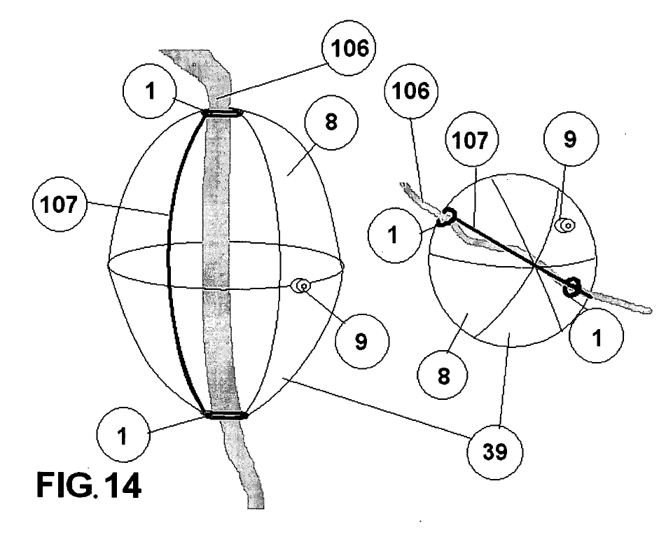

FIG. 14 is an isometric view of the dynamic action implantable decompression chamber 39 embodiment of the present invention. The dynamic action implantable decompression chamber 39 is one embodiment that can be used to enhance tissue deep inside the body. This device has the potential to open collapsed blood vessels or arteries 106, stimulate neurological growth and expand any tissue that it encloses inside the transparent vacuum vessel 8. The dynamic action implantable decompression chamber 39 is made up of a load diffusion seal 1 in combination with a transparent vacuum vessel 8. A bi-directional check vacuum valve 9 is inserted into the transparent vacuum vessel 8 for control of vacuum. The load diffusion seal 1 at either end of the transparent vacuum vessel 8 interfaces with the blood vessel or artery 106. The transparent vacuum vessel 8 is applied via a clamshell methodology along leak proof seam and joint 107. These combined elements allow the embodiment to be implanted within the body and for the transparent vacuum vessel 8 to be connected via vacuum tubing to the outside of the user or patient's body. It envisioned that this dynamic action implantable decompression chamber 39 could be made of materials that safely dissolve within the body so that extraction of the dynamic action implantable decompression chamber 39 after strengthening the blood vessel or artery 106 is not required. It is further envisioned that the dynamic action implantable decompression chamber 39 will be made of material that allows both or either X-rays and magnetic resonance imaging (MRI) emissions through it without hiding the blood vessel or artery 106 tissue inside the dynamic action implantable decompression chamber 39 so the treatment regimen and progress can be monitored. U.S. patent applications having publication numbers 20050107870 and 20050079132 filed by Wang et al. for a “Medical Device with Multiple Coating Layers” and “Medical Device with Low Magnetic Susceptibility” provide thorough examinations related to magnetic resonance imaging.

FIG. 15 is a graphic chart comparing dynamic load diffusion to static load distribution. This computer model simulation clearly establishes that in fact, the dynamic properties of load diffusion significantly reduce the stresses associated upon supporting and surrounding tissues engaged with the load diffusion seal 1 and thus outperform load distribution platforms and other cushioned applications as found in the prior art.

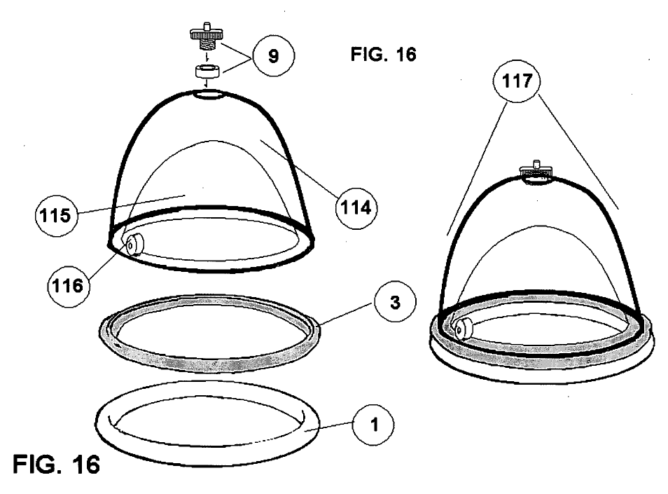

FIG. 16 is an isometric view of the double-chambered decompression device assembly 117 of the present invention. This embodiment of the present invention presents a double chambered decompression device assembly 117 that uses a double dome or chamber to multiply the amount of decompressive energy that may be applied to the living tissue. This embodiment utilizes a substantially similar load diffusion seal 1 in combination with a dynamic energy transfer collar 3. Additionally, a bi-directional check vacuum valve 9 has been installed in the external transparent vacuum vessel 114. In this embodiment, an internal transparent vacuum vessel 115 having a bi-directional check vacuum valve 116 is placed inside of a specialized external transparent vacuum vessel 114, which are engaged through the double chambered decompression device assembly 117. The combination of two vacuum vessels, one inside of the other, with individual check valves engaged with tissue through load diffusion seals 1 protects the external supporting and surrounding tissue while allowing for localized and specialized deep tissue penetration through the large amount of vacuum that can be applied via two inter-deposed vacuum vessels.

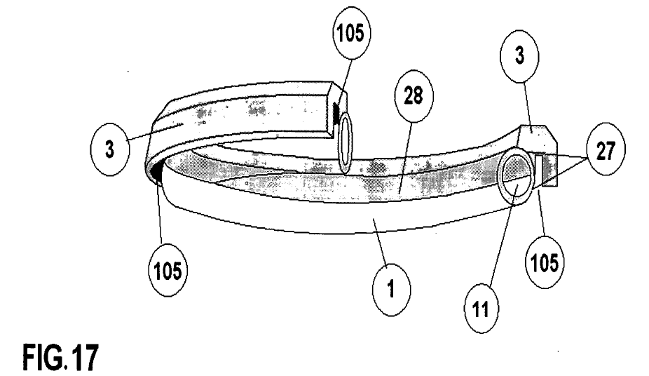

FIG. 17 is an isometric view of the load diffusion seal 1 of the present invention. In this isometric view, the dynamic energy transfer collar 3 is shown in combination with the collar's vessel mating groove 105 to opposing surface contact area 27 and the chamber mating section with the dynamic energy transfer collar 28 all mated positively together to load diffusion seal 1 which encases the fluid filled chamber 11, thus creating the load diffusion seal assembly.

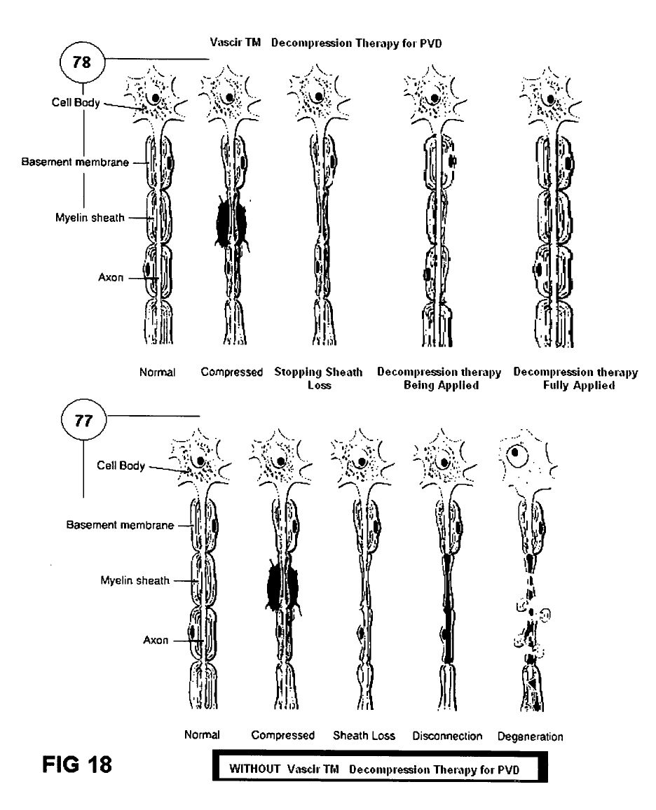

FIG. 18 pictorially illustrates the envisioned effects of Vascir™ decompression therapy on damaged neurological tissues. The nerve tissue damaged and without Vascir™ Therapy 77 which is weak and diseased, and nerve tissue damaged and with Vascir™ Therapy 78 which is healthy and stronger.

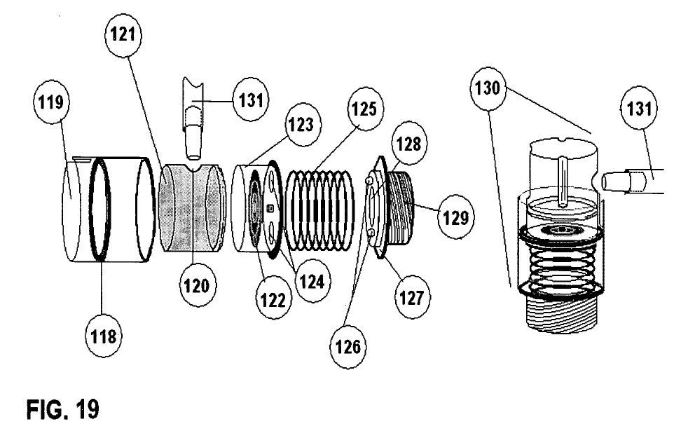

FIG. 19 presents an isometric view of a multi valve assembly 130 that may be used in conjunction with the present invention the purpose of which is to provide a combination check and relief valve which maintains vacuum while allowing the user to manually release the vacuum in the chamber for various purposes including comfort and/or emergency relief. The multi valve assembly 130 via multi valve threaded end 129 is engaged and connected with the vacuum vessel and vacuum tubing with pressure sensitivity for safety 131. Threaded end 129 and multi valve return spring retainer evacuation port 128 in combination with multi valve stop plate 127 cooperate together to provide a flow through pedestal for the multi valve assembly 130. The multi valve assembly 130 allows evacuation of the transparent vacuum vessel 8 via cooperation of multi valve return ring 125, multi valve check valve diaphragm 122 and multi valve check valve housing 123 when a pump is connected and provides suitable down decompression pressure (vacuum) to depress multi valve return spring 125, which allows multi valve return spring retainer with evacuation nubs 126 to engage with multi valve dual purpose inlet exhaust ports evacuation ports 124 and disengage multi valve check valve diaphragm 122 from the multi valve check valve housing 123. This allows air to be evacuated from the transparent vacuum vessel 8. Removal of the pump releases the pressure against the multi valve return spring 125 allowing disengagement of multi valve return spring retainer with evacuation nubs 126 and multi valve dual purpose inlet exhaust ports 124, thereby sealing or closing the valve and holding vacuum in the transparent vacuum vessel 8. To release the vacuum, application of force upon the multi-valve activation button with bleed 120 depresses multi valve return spring 125 which allows multi valve return spring retainer with evacuation nubs 126 to engage with multi valve dual purpose inlet exhaust ports 124 to disengage multi valve check valve diaphragm 122 from the multi valve check valve housing 123 to allow fluid or air to pass back through the multi valve assembly 130 and out vacuum tubing with pressure sensitivity for safety 131. Multi-valve o-ring 118 in cooperation with multi valve button sheath with cut out 119 cooperate with multi valve activation button with bleed 120 with multi valve activation button bleed off channel 121 to seal the multi valve assembly 130 during vacuum operation.

FIG. 20 illustrates how living cellular tissue reacts in the presence of vacuum energy. The expanding and stressing of the very biological structures that hold the cells and cellular matter together. During the decompression process, the normal healthy human cell without vacuum decompression applied 40 and its membrane are temporarily expanded, stretched and thinned, into normal healthy human cell with vacuum decompression applied 41 and normal healthy human cell with vacuum decompression fully applied with thin membrane 42 by the process of deep penetrating decompression, due to and dependent upon the amount of vacuum applied (up to 30 inches of Hg) in the particular chamber being utilized and the result sought. The expected healthy responses are indicated by normal healthy human cell after treatment is oxygen enriched and vibrant 43 and normal healthy human cell after treatment with stronger membrane with more flexibility 44 of this illustration. As illustrated, the normal healthy human cell after treatment with stronger membrane with more flexibility 44 is thicker and more malleable and the normal healthy human cell after treatment is oxygen enriched and vibrant 43 is oxygenated and has an abundance of nutrients and life supporting blood.

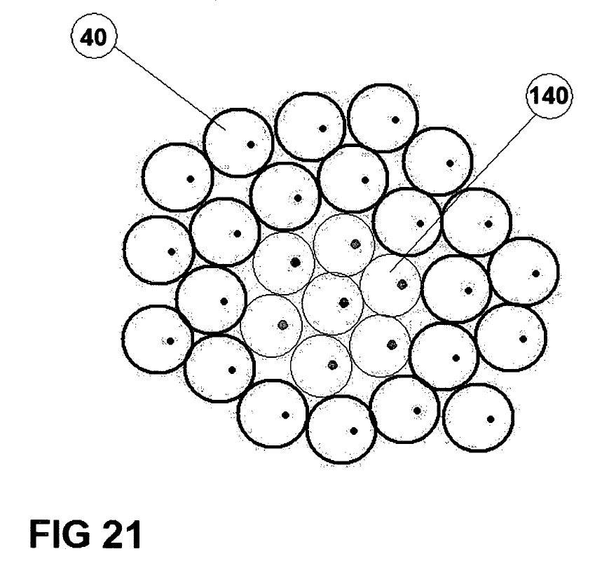

FIG. 21 is an illustration of tissue having cancerous cells in combination with non-cancerous cells. Normal healthy human cells without vacuum decompression applied 40 can and do sometimes surround or have incorporated within and/or around them cancer cells in otherwise healthy human without vacuum decompression applied 140. These cancer cells in otherwise healthy human without vacuum decompression applied 140 can be cancerous and/or tumorous in nature. This illustration is a general representation of a cluster of cells. The cancer cells in otherwise healthy human without vacuum decompression applied 140 are surrounded by the normal healthy human cells without vacuum decompression applied 40. In this state, the cells show initial signs of decompressive energy (expanding) being applied.

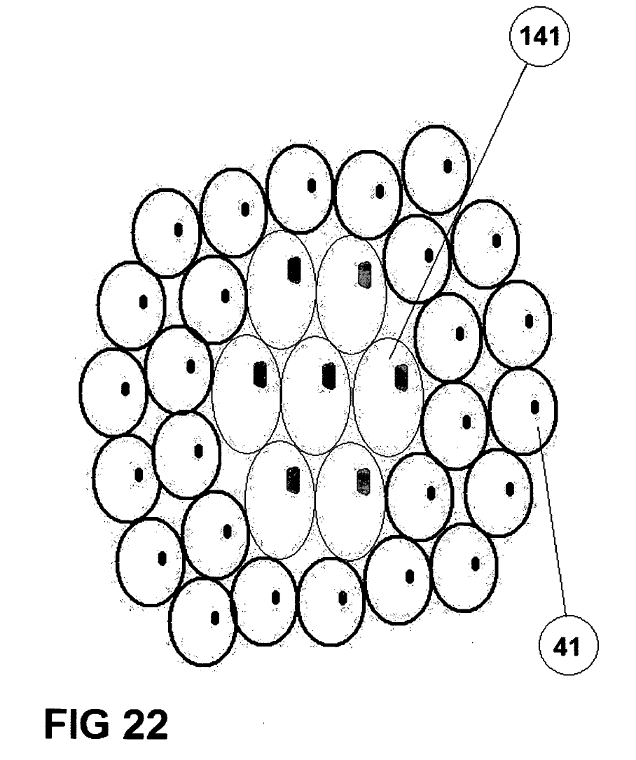

FIG. 22 is an illustration of tissues reacting to decompressive energy having cancer cells in otherwise healthy human with vacuum decompression being applied 141 in combination with normal healthy human cell with vacuum decompression being applied 41. Normal healthy human cell with vacuum decompression being applied 41 with their stronger membranes can accommodate these decompressive forces by enlarging and/or expanding in size. The cell membranes of healthy cells being stronger and more resilient are able to stretch, and become thinner without the cell membranes of the normal healthy human cell with vacuum decompression being applied 41 reaching the point of rupturing or breaking. Cancer cells in otherwise healthy human with vacuum decompression being applied 141, however, have a membrane that is thinner which results in a more dramatic response to the decompressive forces being applied to the cancer cells in otherwise healthy human with vacuum decompression being applied 141.

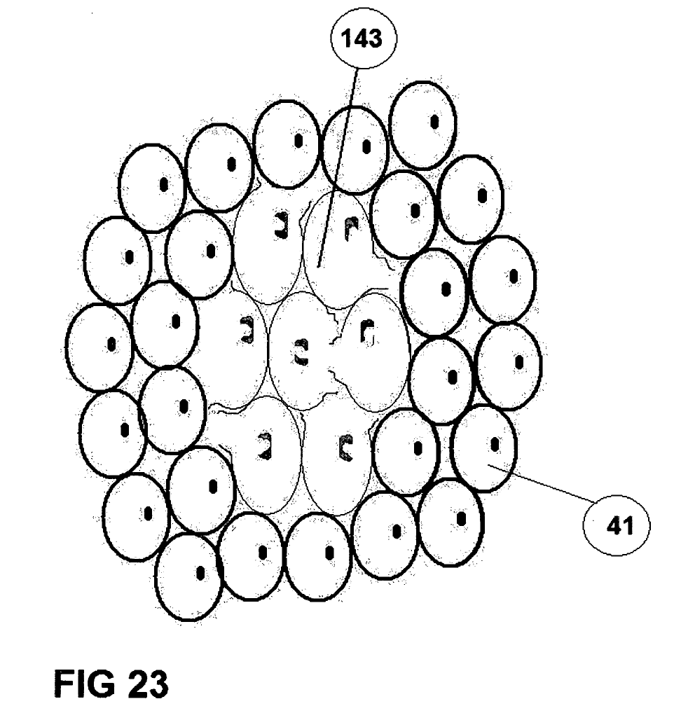

As illustrated in FIG. 23, continuing to apply decompressive energy to the cancer cells in otherwise healthy human with vacuum decompression being applied 143, which have a membrane that is thinner and more responsive to the decompressive forces applied, results in rupture and destruction of the cancer cells in otherwise healthy human with vacuum decompression being applied 143, while the stronger and more resilient membrane of the normal healthy human cell with vacuum decompression being applied 41 allows them to stretch and become thinner without reaching the point of breaking or rupturing. The method and apparatus herein may also be used with a pharmacological composition selected for membrane rupture of cancer cell in otherwise healthy human with vacuum decompression being applied 143.

One example of beneficial compositions is generally known as “chemotherapy,” which may include a combination of the following drugs cyclophosphamide, hydroxydaunorubicin (also sometimes known as adriamycin or doxorubicin) and vincristine. Other pharmacological compositions beneficial to membrane rupture of cancer cell in otherwise healthy human with vacuum decompression being applied 143 may also be used in combination with decompressive energy. The application of decompressive energy to cancer cells in otherwise healthy human without vacuum decompression applied 140 effectively increases the permeability of the cancer cells in otherwise healthy human without vacuum decompression applied 140 membranes, increasing the efficacy of the cancer pharmacological composition thereby aiding in destruction of the cancer cells in otherwise healthy human without vacuum decompression applied 140. Use and delivery of these pharmacological compositions may be further improved if used in combination with nano-devices that rupture or are actuated when they come in to contact with or pass through the decompressive energy gradient 137, thus delivering their pharmacological payload directly to the area needed to be treated. This improved delivery and targeting of the pharmacological compositions to the treated tissues is critical for effective treatment. As those skilled in the arts will appreciate, nano devices containing pharmacological compositions may also be introduced into the treated tissue directly or indirectly in one of the following four (4) ways, or through a combination of them including, intravenous (IV) infusion, by pill, by injection or shot, and/or through intrathecal and intraventricular injection.

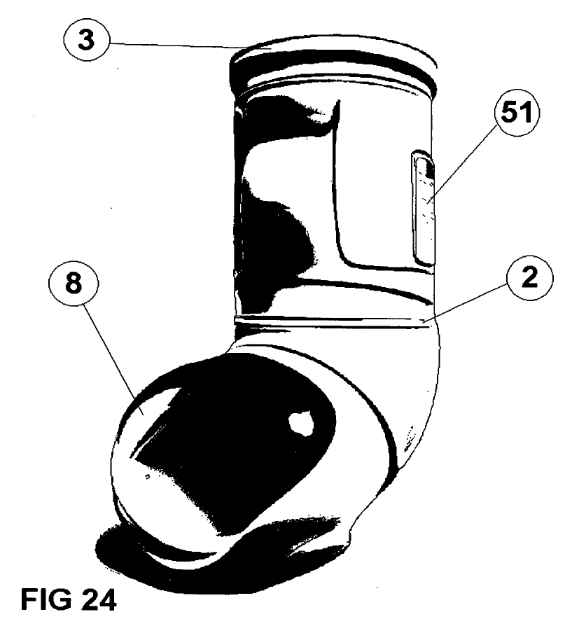

FIG. 24 is another embodiment of FIG. 2 to surround the patient's foot. Similar to FIG. 2, the dynamic energy transfer collar 3 is attached to the vacuum vessel 2. This illustration also embodies the incorporation of the smart chip controlled vacuum charging system device assembly 51 for autonomous operation and fulfillment of the medical prescription on usage, and the transparent vacuum vessel 8, allowing visual examination of the tissue being treated.

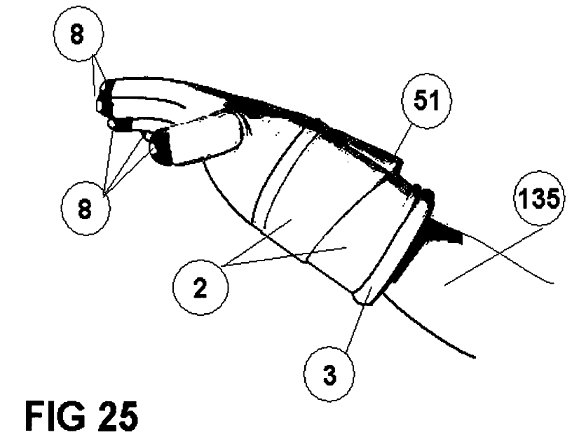

FIG. 25 is another embodiment of FIG. 3, a hand and upper extremity unit that encapsulates the patient's upper extremity soft and/or permeable tissue 135. The hand and upper extremity unit consists of the dynamic energy transfer collar 3 and the vacuum vessel 2. This illustration also embodies the incorporation of the smart chip controlled vacuum charging system device assembly 51 for autonomous operation and fulfillment of the medical prescription on usage, and the transparent vacuum vessel 8, allowing visual examination of the tissue being treated.

The following references are also cited in support of the present application:
1. Hirsch A T, Munnings F. Intermittent claudication. Physician Sports Med 1993; 21(6).
2. Lindgarde F, Jelnes R, B jorkman H, et al. Conservative drug treatment in patients with moderately severe chronic occlusive peripheral arterial disease. Circulation 1989; 80: 1549-56.
3. AD—Department of Epidemiology, Graduate School of Public Health, University of Pittsburgh, Pa., USA. Greene, D A, Feldman, E L, Stevens, M J, et al. Diabetic neuropathy. In: Diabetes Mellitus, Porte, D, Sherwin, R, Rifkin, H (Eds), Appleton Lange, East Norwalk, Conn., 1995.
4. Pirart, J. Diabetes mellitus and its degenerative complications: A prospective study of 4,400 patients observed between 1947 and 1973. Diabetes Care 1978; 1:168.
5. TI—A multicentre study of the prevalence of diabetic peripheral neuropathy in the United Kingdom hospital clinic population. AU—Young M J; Boulton A J; MacLeod A F; Williams D R; Sonksen P H. SO—Diabetologia 1993 February; 36(2):150-4.
6. TI—Epidemiological correlates of diabetic neuropathy. Report from Pittsburgh Epidemiology of Diabetes Complications Study. AU—Maser R E; Steenkiste A R; Dorman J S; Nielsen V K; Bass E B; Manjoo Q; Drash A L; Becker D J; Kuller L H; Greene D A; et al. SO—Diabetes 1989 November; 38(11):1456-61.
7. Report and recommendations of the San Antonio Conference on Diabetic Neuropathy. Diabetes 1988; 37:1000.
8. TI—Incidence of distal symmetric (sensory) neuropathy in NIDDM. The San Luis Valley Diabetes Study. AU—Sands M L; Shetterly S M; Franklin G M; Hamman R F. SO—Diabetes Care 1997 March; 20(3):322-9. AD—Department of Preventive Medicine and Biometrics, University of Colorado School of Medicine, Denver 80262, USA.
9. TI—Hypertension as a risk factor for diabetic neuropathy: a prospective study.
AU—Forrest K Y; Maser R E; Pambianco G; Becker D J; Orchard T J SO—Diabetes 1997 April; 46(4):665-70.
10. TI—The contribution of non-insulin-dependent diabetes to lower-extremity amputation in the community. AU—Humphrey L L; Palumbo P J; Butters M A; Hallett J W Jr; Chu C P; O'Fallon W M; Ballard D J SO—Arch Intern Med 1994 Apr. 25; 154(8):885-92.

It should be noted that the present invention is not limited to the specific embodiments pictured and described herein, but is intended to apply to apparati and methods employing decompressive energy to stimulate tissue growth, enhancement, circulation and/or selective destruction of diseased cells, particularly those having malignant tendencies. Modifications and alterations from the described embodiments will occur to those skilled in the art without departure from the spirit and scope of the present invention.

What is claimed is:

1. An apparatus for delivering decompressive energy to soft tissues to stimulate cellular expansion through deep penetration of said applied decompressive energy to said soft tissues to stimulate a predetermined reaction to application of said decompressive energy comprising:
a. a vessel having an open end and adapted to encompass the soft tissue to be stimulated;
b. a source of decompressive energy in communication with said vessel; and,
c. a flexible mass affixed to said open end of said vessel to absorb the pressure exerted by delivery of said decompressive energy to said soft tissue, thereby acting as a seal and force diffuser between said vessel and the soft tissue adjacent the periphery of said vessel.

2. The apparatus in accordance with claim 1, wherein a compressive wrap is placed on said soft tissue to be treated.

3. The apparatus in accordance with claim 1, wherein said source of decompressive energy also includes a control mechanism to control the level of the decompressive energy applied to said soft tissue.

4. The apparatus in accordance with claim 3, wherein said control mechanism includes a computer control system for controlling treatment of said tissue.

5. The apparatus in accordance with claim 1, wherein said control mechanisms allows the level of decompressive energy oscillate during application of said decompressive energy to said tissue.

6. The apparatus in accordance with claim 1, further including sensors for controlling treatment of said soft tissue.

7. The apparatus in accordance with claim 1, wherein repeated applications to said tissue treated for peripheral vascular disease stimulates tissue genesis.

8. The apparatus in accordance with claim 1, wherein said vessel will withstand a vacuum of 30 inches of Hg.

9. The apparatus in accordance with claim 1, wherein said soft tissue is treated for peripheral vascular disease.

10. The apparatus in accordance with claim 1, wherein a pharmacological composition beneficial to said soft tissue is introduced into said soft tissue in combination with said decompressive energy.

11. An apparatus for applying vacuum to tissue having a vascular system to expand and stimulate said vascular system comprising:
a. a vessel having an open end and adapted to encompass the tissue to be treated;
b. a source of vacuum connected to said vessel; and
c. a flexible mass affixed to the open end of said vessel to absorb the pressure exerted by said vacuum, thereby acting as a seal and force diffuser between the vessel and the tissue adjacent the periphery of said vessel.

12. The apparatus in accordance with claim 11, wherein said vessel has a shape generally conforming to the shape of the tissue to be treated.

13. The apparatus in accordance with claim 11, wherein said tissue is treated for peripheral vascular disease.

14. The apparatus in accordance with claim 13, wherein said tissue is treated for peripheral vascular disease and repeated use of said apparatus increases vascular elasticity.

15. The apparatus in accordance with claim 13, wherein repeated applications to said tissue treated for peripheral vascular disease stimulates tissue genesis.

16. The apparatus in accordance with claim 14, wherein repeated applications to said tissue treated for peripheral vascular disease increases the elasticity of the vascular system of said treated tissue.

17. The apparatus in accordance with claim 16, wherein said vessel has a shape generally conforming to the shape of the tissue to be treated.

18. The apparatus in accordance with claim 11, wherein said vessel has a volume greater than the volume of tissue to be treated for peripheral vascular disease.

19. The apparatus in accordance with claim 18, wherein said vessel has a shape, which is varied to control the shape of the tissue to be treated for peripheral vascular disease.

20. The apparatus in accordance with claim 11, wherein said vessel is dome-shaped having a periphery surrounding the tissue to be treated for peripheral vascular disease.

21. The apparatus in accordance with claim 11, wherein said vessel has an opening separate from said open end for connection to said source of vacuum.

22. The apparatus in accordance with claim 11, wherein said flexible mass includes an air pocket.

23. The apparatus in accordance with claim 11, wherein said flexible mass includes a fluid filled pocket.

24. The apparatus in accordance with claim 11, wherein said flexible mass includes a pocket filled with energy absorbing material.

25. The apparatus in accordance with claim 22, wherein said mass and said air pocket are substantially aligned with the centerline of the periphery of the open end of said vessel.

26. The apparatus in accordance with claim 25, wherein said periphery of the open end of said vessel includes flanges on both surfaces of said vessel at angles to the centerline of said periphery.

27. The apparatus in accordance with claim 26, wherein said flanges have an arcuate configuration.

28. The apparatus in accordance with claim 27, wherein said arcuate configuration is convex with respect to the periphery of said vessel.

29. The apparatus in accordance with claim 25, wherein said flange applies the force of the vacuum to the flexible mass and said air pocket to substantially diffuse the force of the vacuum applied at the base of the flexible mass affixed to said vessel.

30. The apparatus in accordance with claim 11, wherein said connection between said vacuum source and said vessel, includes a valve mechanism.

31. The apparatus in accordance with claim 30, wherein said valve mechanism includes a check valve.

32. The apparatus in accordance with claim 31, wherein said valve mechanism includes a relief valve.

33. The apparatus in accordance with claim 11, wherein said vessel will withstand a vacuum of 30 inches of Hg.

34. The apparatus in accordance with claim 11, wherein said source of vacuum includes a control mechanism to control the value of the vacuum provided.

35. The apparatus in accordance with claim 34, wherein said control mechanisms will control the vacuum from 0.00001 inches of Hg to a maximum of 30 inches of Hg to be applied to said vessel.

36. The apparatus in accordance with claim 35, wherein said control mechanisms allow the level of vacuum to be oscillated during application of said vacuum to said tissue.

37. The apparatus in accordance with claim 36, wherein said tissue is treated for peripheral vascular disease and repeated use of said apparatus benefits vascular health.

38. The apparatus in accordance with claim 37, wherein repeated use of said apparatus benefits tissue health as measured by variables selected from the group consisting of vascular elasticity, vascular strength, hydrostatic pressure on arterial and venous ends of the capillary, oncotic blood and tissue pressure (Pob and Pot), pore radius in the capillary, the number of pores in the capillary per millimeter, the amount of the liquid released from the capillary, gas diffusion coefficient, time of erythrocyte movement in the capillary, speed of oxygen consumption, characteristic time of diffusion, increased number and diameter of hydraulic pores and combinations thereof.

39. The apparatus in accordance with claim 36, wherein said tissue is treated for peripheral vascular disease and repeated use of said apparatus benefits vascular health.

40. The apparatus in accordance with claim 37, wherein repeated use of said apparatus benefits tissue health as measured by variables selected from the group consisting of vascular elasticity, vascular strength, hydrostatic pressure on arterial and venous ends of the capillary, oncotic blood and tissue pressure (Pob and Pot), pore radius in the capillary, the number of pores in the capillary per millimeter, the amount of the liquid released from the capillary, gas diffusion coefficient, time of erythrocyte movement in the capillary, speed of oxygen consumption, characteristic time of diffusion, increased number and diameter of hydraulic pores and combinations thereof.

41. The apparatus in accordance with claim 34 wherein a compressive wrap is placed between said tissue to be treated and said vacuum source.

42. The apparatus in accordance with claim 36, wherein a pharmacological composition beneficial to increasing vascular elasticity and permeability is introduced into said tissue in combination with said vacuum.

43. The apparatus in accordance with claim 36, wherein a pharmacological composition beneficial in vascular angiogenesis and vasculogenesis is introduced into said tissue in combination with said vacuum.

44. The apparatus in accordance with claim 40, wherein said pharmacological composition beneficial to increasing vascular elasticity, strength and or permeability is selected from the group consisting of peripheral vasodilators, anticoagulants, beta blockers, combined alpha and beta blockers, central alpha agonists, peripheral alpha-1 blockers, angiotensin converting enzyme (ACE) inhibitors, calcium channel blockers, fenoldopam and combinations thereof.

45. The apparatus in accordance with claim 41, wherein said pharmacological composition beneficial to increasing vascular angiogenesis and or vasculogenesis is selected from the group consisting of peripheral vasodilators, anticoagulants, beta blockers, combined alpha and beta blockers, central alpha agonists, peripheral alpha-1 blockers, angiotensin converting enzyme (ACE) inhibitors, calcium channel blockers, fenoldopam and or any other pharmacological compounds or combinations thereof.

46. The apparatus in accordance with claim 40, wherein said pharmacological composition is encapsulated in a nano device for improved delivery of said pharmacological compositions to said treated tissues.

47. The apparatus in accordance with claim 41, wherein said pharmacological composition is encapsulated in a nano device for improved delivery of said pharmacological compositions to said treated tissues.

48. The apparatus in accordance with claim 44, wherein said nano device is actuated by decompressive forces for improved delivery of said pharmacological compositions to said treated tissues.

49. The apparatus in accordance with claim 45, wherein said nano device is actuated by decompressive forces for improved delivery of said pharmacological compositions to said treated tissues.

50. The apparatus in accordance with claim 11, further including sensors for controlling treatment of said tissue.

51. The apparatus in accordance with claim 47 wherein said sensors are selected from the group consisting of oxygen, light, temperature, atmospheric water vapor content, vacuum and combinations thereof.

52. The apparatus in accordance with claim 11, further including a computer control system for controlling treatment of said tissue.

53. The apparatus in accordance with claim 49 wherein said sensors are selected from the group consisting of oxygen, light, temperature, blood flow rate, vascular elasticity, atmospheric water vapor content, vacuum level and combinations thereof.

54. The apparatus in accordance with claim 50 wherein said computer control system includes a removable memory module able to store patient data and data collected during tissue treatment.

55. The apparatus in accordance with claim 11 wherein a compressive wrap is placed around tissue to be treated.

56. The apparatus in accordance with claim 53 wherein the functional actions of said compressive wrap are selected from the group consisting of limiting tissue expansion, preventing tissue edema, allowing higher levels of vacuum, allowing deeper penetration of vacuum forces, alleviating patient discomfort during tissue treatment and allowing adjustment of compressive forces against said tissue and combinations thereof.

57. The apparatus in accordance with claim 54 wherein said compressive wrap is selectively placed over a portion of said tissue to be treated.

58. The apparatus in accordance with claim 55 wherein a second vessel having a force diffusion seal is placed over said tissue within said first vessel during treatment of said tissue.

59. The apparatus in accordance with claim 56 wherein said second vessel is selectively placed over a portion of said tissue to be treated.

60. The apparatus in accordance with claim 12, wherein said vessel is shaped to treat the lower extremities of a human.

61. The apparatus in accordance with claim 58, wherein said vessel is shaped enclose a human foot.

62. The apparatus in accordance with claim 59, further including a computer control system for controlling treatment of said tissue.

63. The apparatus in accordance with claim 60, wherein said vessel is shaped enclose and treat the toes of a human foot.

64. The apparatus in accordance with claim 12, wherein said vessel is shaped to enclose and treat a human hand.

65. The apparatus in accordance with claim 62, wherein said vessel is shaped to enclose and treat the digits of a human hand.

66. An apparatus for treatment of tissue afflicted with circulatory diseases by application of vacuum forces comprising:
a. a vessel having walls and defining a first and second openings, said first opening adapted to encompass the tissue to be treated;
b. a source of vacuum having a comfort control valve and vacuum control unit with a plurality of settings connected to said vessel;
c. a mass of elastic material, said mass of elastic material further comprising:
i. an inner radius and outer radius, said inner radius forming a seal with said tissue while allowing said tissue to move in relation to said inner radius;
ii. a fluid pocket circumferentially positioned within said elastic mass;
d. a collar positioned at the perimeter of said first opening, said collar further comprising:
iii. an inner flange affixed to said outer radius of said elastic material, said inner flange having an arcuate shape complimentary to said outer radius;
iv. an outer flange having a top and a bottom portion, said bottom portion extending downwardly along vessel wall to terminate substantially in line with said inner radius of said mass of elastic material and wherein said top portion extends past said opening of said vessel to terminate at a right angle with respect to said vessel wall creating a load diffusion seal to diffuse both said applied vacuum forces and the forces generated between the interior of said vessel and said tissue which said vessel encompasses;
e. a valve assembly comprising a comfort control valve, a check valve and a relief valve, said valve assembly positioned at said second vessel opening; and,
f. a section of tubing connecting said valve assembly and said source of vacuum wherein said control valve limits the vacuum from 0.000001 mm Hg to a maximum of >30 Hg to said vessel.

67. The apparatus in accordance with claim 66, wherein said inner flange is embedded in said outer radius of said elastic material.

68. The apparatus in accordance with claim 66, wherein said flanges transmit a plurality of forces generated between said vessel and said load diffusion seal and said fluid pocket to substantially diffuse the force of the vacuum applied to the tissue in contact with said load diffusion seal.

69. The apparatus in accordance with claim 68, wherein said plurality of forces are dynamic.

70. The apparatus in accordance with claim 68, wherein the diffusion of forces is dynamic.

71. The apparatus in accordance with claim 66, wherein said elastic mass containing said fluid pocket and said inner and outer flanges form one continuous unit.

72. The apparatus in accordance with claim 66, wherein said fluid pocket contains air.

73. The apparatus in accordance with claim 66, wherein said vacuum pump assembly includes a source of power.

74. The apparatus in accordance with claim 66, wherein said vacuum pump assembly is manually operated.

75. The apparatus in accordance with claim 66, wherein said collar has first and second radial portions allowing cooperative assembly.

76. The apparatus in accordance with claim 75, wherein said vessel has a seam perpendicular said collar and parallel said vessel walls, said seam allowing cooperative assembly of a first and second portion of vessel.

77. The apparatus in accordance with claim 75, wherein said first and second radial portions of said collar interlock.

78. The apparatus in accordance with claim 77, wherein said first and second portions of vessel interlock at said vessel seam.

79. The apparatus in accordance with claim 66, wherein said outer flange bottom portion terminates at the intersection of the tangent line at the interface between said outer radius of said elastic material and said inner flange.

80. The apparatus in accordance with claim 76, wherein said outer flange bottom portion terminates at the intersection of the tangent line at the interface between said outer radius of said elastic material and said inner flange.

81. The apparatus in accordance with claim 77, wherein said fluid pocket has a center point and said top portion of said outer flange terminates at the intersection of the line bisecting the arc of said elastic material outer radius and said center point of said fluid pocket.

82. The apparatus in accordance with claim 66, wherein said tissue is treated for peripheral vascular disease and repeated use of said apparatus increases vascular elasticity.

83. The apparatus in accordance with claim 66, wherein repeated applications to said tissue treated for peripheral vascular disease stimulates tissue genesis.

84. The apparatus in accordance with claim 76, wherein repeated applications to said tissue treated for peripheral vascular disease increases the elasticity of the vascular system of said treated tissue.

85. The apparatus in accordance with claim 66, wherein a pharmacological composition beneficial to increasing vascular elasticity and permeability is introduced into said tissue in combination with said vacuum.

86. The apparatus in accordance with claim 66, wherein a pharmacological composition beneficial in vascular angiogenesis is introduced into said tissue in combination with said vacuum.

87. The apparatus in accordance with claim 85, wherein said pharmacological composition beneficial to increasing vascular elasticity and permeability is selected from the group consisting of peripheral vasodilators, anticoagulants, beta blockers, combined alpha and beta blockers, central alpha agonists, peripheral alpha-1 blockers, angiotensin converting enzyme (ACE) inhibitors, calcium channel blockers, fenoldopam, purified nucleic acid sequences encoding GTP cyclohydrolase (GTPCH) polypeptide and or any other pharmacological compounds or combinations thereof.

88. The apparatus in accordance with claim 87, wherein said pharmacological composition beneficial to increasing vascular angiogenesis is selected from the group consisting of peripheral vasodilators, anticoagulants, beta blockers, combined alpha and beta blockers, central alpha agonists, peripheral alpha-1 blockers, angiotensin converting enzyme (ACE) inhibitors, calcium channel blockers, fenoldopam, purified nucleic acid sequences encoding GTP cyclohydrolase (GTPCH) polypeptide and or any other pharmacological compounds or combinations thereof.

89. The apparatus in accordance with claim 86, wherein said pharmacological composition is encapsulated in a nano device for improved delivery of said pharmacological compositions to said treated tissues.

90. The apparatus in accordance with claim 87, wherein said pharmacological composition is encapsulated in a nano device for improved delivery of said pharmacological compositions to said treated tissues.

91. The apparatus in accordance with claim 88, wherein said nano device is actuated by decompressive forces for improved delivery of said pharmacological compositions to said treated tissues.

92. The apparatus in accordance with claim 87, wherein said nano device is actuated by decompressive forces for improved delivery of said pharmacological compositions to said treated tissues.

93. The apparatus in accordance with claim 66 further including a computer control system for controlling treatment of said tissue.

94. The apparatus in accordance with claim 93 further including sensors for controlling treatment of said tissue.

95. The apparatus in accordance with claim 94 wherein said sensors are selected from the group consisting of oxygen, light, temperature, atmospheric water vapor content, vacuum and combinations thereof.

96. The apparatus in accordance with claim 93 wherein said computer control system includes a removable memory module able to store patient data and data collected during tissue treatment.

97. The apparatus in accordance with claim 66 wherein a compressive wrap is placed around tissue to be treated.

98. The apparatus in accordance with claim 97 wherein said compressive wrap is selectively placed over a portion of said tissue to be treated.

99. The apparatus in accordance with claim 66 wherein a second vessel having a force diffusion seal is placed over said tissue within said first vessel during treatment of said tissue.

100. The apparatus in accordance with claim 99 wherein said second vessel is selectively placed over a portion of said tissue to be treated.

101. The apparatus in accordance with claim 100, wherein said vessel is shaped to treat the lower extremities of a human.

102. The apparatus in accordance with claim 101, wherein said vessel is shaped to enclose a human foot.

103. The apparatus in accordance with claim 102, wherein said vessel is shaped to segregate treatment of said toes of a human foot.

104. The apparatus in accordance with claim 66, wherein said vessel is shaped to enclose and treat a human hand.

105. The apparatus in accordance with claim 104, wherein said vessel is shaped to enclose and treat the digits of a human hand.

106. An apparatus for delivering decompressive energy to tissue having a vascular system to expand and stimulate said vascular system, either directly or indirectly, comprising:
a. a vessel having an open end and adapted to encompass the tissue to be treated;
b. a source of decompressive energy in communication with said vessel; and,
c. a flexible mass affixed to the open end of said vessel to absorb the pressure exerted by delivery of said decompressive energy to said tissue, thereby acting as a seal and force diffuser between the vessel and tissue adjacent the periphery of said vessel.

107. The apparatus in accordance with claim 106, wherein said vessel has a shape generally conforming to the shape of the tissue to be treated.

108. The apparatus in accordance with claim 106, wherein said tissue is treated for peripheral vascular disease.

109. The apparatus in accordance with claim 108, wherein said tissue is treated for peripheral vascular disease and repeated use of said apparatus increases vascular elasticity.

110. The apparatus in accordance with claim 108, wherein repeated applications to said tissue treated for peripheral vascular disease stimulates tissue genesis.

111. The apparatus in accordance with claim 109, wherein repeated applications to said tissue treated for peripheral vascular disease increases the elasticity of the vascular system of said treated tissue.

112. The apparatus in accordance with claim 111, wherein said vessel has a shape generally conforming to the shape of the tissue to be treated.

113. The apparatus in accordance with claim 106, wherein said vessel has a volume greater than the volume of tissue to be treated for peripheral vascular disease.

114. The apparatus in accordance with claim 113, wherein said vessel has a shape, which is varied to control the shape of the tissue to be treated for peripheral vascular disease.

115. The apparatus in accordance with claim 106, wherein said vessel is dome-shaped having a periphery surrounding the tissue to be treated for peripheral vascular disease.

116. The apparatus in accordance with claim 106, wherein said vessel has an opening separate from said open end for connection to said source of decompressive energy.

117. The apparatus in accordance with claim 106, wherein said flexible mass includes an air pocket.

118. The apparatus in accordance with claim 106, wherein said flexible mass includes a fluid filled pocket.

119. The apparatus in accordance with claim 106, wherein said flexible mass includes a pocket filled with energy absorbing material.

120. The apparatus in accordance with claim 117, wherein said mass and said air pocket are substantially aligned with the centerline of the periphery of the open end of said vessel.

121. The apparatus in accordance with claim 120, wherein said periphery of the open end of said vessel includes flanges on both surfaces of said vessel at angles to the centerline of said periphery.

122. The apparatus in accordance with claim 121, wherein said flanges have an arcuate configuration.

123. The apparatus in accordance with claim 122, wherein said arcuate configuration is convex with respect to the periphery of said vessel.

124. The apparatus in accordance with claim 120, wherein said flange applies the force of the decompressive energy to the flexible mass and said air pocket to substantially diffuse the force of the decompressive energy applied at the base of the flexible mass affixed to said vessel.

125. The apparatus in accordance with claim 106, wherein said connection between said decompressive energy source and said vessel, includes a valve mechanism.

126. The apparatus in accordance with claim 125, wherein said valve mechanism includes a check valve.

127. The apparatus in accordance with claim 126, wherein said valve mechanism includes a relief valve.

128. The apparatus in accordance with claim 106, wherein said vessel will withstand a vacuum of 30 inches of Hg.

129. The apparatus in accordance with claim 106, wherein said source of decompressive energy includes a control mechanism to control the level of the decompressive energy provided.

130. The apparatus in accordance with claim 129, wherein said control mechanisms will control the decompressive energy level from 0.00001 inches of Hg to a maximum of 30 inches of Hg to be applied to said vessel.

131. The apparatus in accordance with claim 130, wherein said control mechanisms allow the level of decompressive energy to oscillate up or down the decompressive gradient in a random and or purposeful manner during application of said vacuum to said tissue.

132. The apparatus in accordance with claim 131, wherein said tissue is treated for peripheral vascular disease and repeated use of said apparatus increases vascular elasticity and health.

133. The apparatus in accordance with claim 132, wherein repeated applications to said tissue treated for peripheral vascular disease stimulates tissue genesis.

134. The apparatus in accordance with claim 132, wherein repeated applications to said tissue treated for peripheral vascular disease increases the elasticity of the vascular system of said treated tissue.

135. The apparatus in accordance with claim 131, wherein a pharmacological composition beneficial to increasing vascular elasticity and permeability is introduced into said tissue in combination with said decompressive therapy.

136. The apparatus in accordance with claim 131, wherein a pharmacological composition beneficial in vascular angiogenesis is introduced into said tissue in combination with said decompressive therapy.

137. The apparatus in accordance with claim 135, wherein said pharmacological composition beneficial to increasing vascular elasticity, strength and or permeability is selected from the group consisting of peripheral vasodilators, anticoagulants, beta blockers, combined alpha and beta blockers, central alpha agonists, peripheral alpha-1 blockers, angiotensin converting enzyme (ACE) inhibitors, calcium channel blockers, fenoldopam, purified nucleic acid sequences encoding GTP cyclohydrolase (GTPCH) polypeptide and or any other pharmacological compounds or combinations thereof.

138. The apparatus in accordance with claim 136, wherein said pharmacological composition beneficial to increasing vascular angiogenesis is selected from the group consisting of peripheral vasodilators, anticoagulants, beta blockers, combined alpha and beta blockers, central alpha agonists, peripheral alpha-1 blockers, angiotensin converting enzyme (ACE) inhibitors, calcium channel blockers, fenoldopam, purified nucleic acid sequences encoding GTP cyclohydrolase (GTPCH) polypeptide and or any other pharmacological compounds or combinations thereof.

139. The apparatus in accordance with claim 135, wherein said pharmacological composition is encapsulated in a nano device for improved delivery of said pharmacological compositions to said treated tissues.

140. The apparatus in accordance with claim 136, wherein said pharmacological composition is encapsulated in a nano device for improved delivery of said pharmacological compositions to said treated tissues.

141. The apparatus in accordance with claim 139, wherein said nano device is actuated by decompressive energy for improved delivery of said pharmacological compositions to said treated tissues.

142. The apparatus in accordance with claim 140, wherein said nano device is actuated by decompressive forces for improved delivery of said pharmacological compositions to said treated tissues.

143. The apparatus in accordance with claim 106, further including sensors for controlling treatment of said tissue.

144. The apparatus in accordance with claim 142, wherein said sensors are selected from the group consisting of oxygen, ultrasound, temperature, atmospheric water vapor content, vacuum and combinations thereof.

145. The apparatus in accordance with claim 106, further including a computer control system for controlling treatment of said tissue.

146. The apparatus in accordance with claim 144, wherein said sensors are selected from the group consisting of oxygen, ultrasound, temperature, blood flow rate, vascular elasticity, atmospheric water vapor content, vacuum level and combinations thereof.

147. The apparatus in accordance with claim 145, wherein said computer control system includes a removable memory module able to store patient data and data collected during tissue treatment.

148. The apparatus in accordance with claim 106, wherein a compressive wrap is placed around tissue to be treated.

149. The apparatus in accordance with claim 148, wherein said compressive wrap applies positive pressure to said treated tissue.

150. The apparatus in accordance with claim 149, wherein said compressive wrap limits tissue expansion during said tissue treatment.

151. The apparatus in accordance with claim 150, wherein said compressive wrap limits edema during treatment of said tissue.

152. The apparatus in accordance with claim 151, wherein said compressive wrap facilitates increased penetration of decompressive energy.

153. The apparatus in accordance with claim 152, wherein said compressive wrap reduces patient discomfort during decompressive tissue therapy.

154. The apparatus in accordance with claim 153, wherein the positive pressure applied by said compressive wrap may be adjusted.

155. The apparatus in accordance with claim 148, wherein said compressive wrap is selectively placed over a portion of said tissue to be treated.

156. The apparatus in accordance with claim 106, wherein a second vessel having a force diffusion seal is placed over said tissue within said first vessel during treatment of said tissue.

157. The apparatus in accordance with claim 156, wherein said second vessel is selectively placed over a portion of said tissue to be treated.

158. The apparatus in accordance with claim 107, wherein said vessel is shaped to treat the lower extremities of a human.

159. The apparatus in accordance with claim 158, wherein said vessel is shaped enclose a human foot.

160. The apparatus in accordance with claim 159, further including a computer control system for controlling treatment of said tissue.

161. The apparatus in accordance with claim 160, wherein said vessel is shaped to enclose and treat the toes of a human foot.

162. The apparatus in accordance with claim 107, wherein said vessel is shaped to enclose and treat a human hand.

163. The apparatus in accordance with claim 162, wherein said vessel is shaped to enclose and treat the digits of a human hand.

164. The apparatus in accordance with claim 66, wherein said vessel is shaped to encompass a human breast.

165. The apparatus in accordance with claim 165, wherein said vessel is shaped to encompass a human breast.

166. The apparatus in accordance with claim 66, wherein said vessel is sized and shaped for insertion within the body.

167. The apparatus in accordance with claim 166, wherein an ultrasonic transducer is placed inside the vessel to monitor stimulation of said tissues.

168. The apparatus in accordance with claim 1, wherein repeated applications to said tissue treated stimulates a positive immune system response.

169. The apparatus in accordance with claim 1, wherein an ultrasonic transducer is placed inside the vessel to monitor stimulation of said tissues.

170. The apparatus in accordance with claim 1, said vessel may be placed inside the body to stimulate a predetermined reaction to said application of decompressive energy.
